(19) United States
(12) Patent Application Publication
(10) Pub. No.: US 2024/0065536 A1
(43) Pub. Date: Feb. 29, 2024

(54) WEIGHTED MANIFOLD FOR AN ENDOSCOPE

(71) Applicant: Boston Scientific Scimed, Inc., Maple Grove, MN (US)

(72) Inventors: Ryan V. Wales, Northborough, MA (US); Colby Harris, Norfolk, MA (US); Paul Smith, Smithfield, RI (US); Kurt Nicholas Robakiewicz, Upton, MA (US); John B. Golden, Norton, MA (US)

(73) Assignee: Boston Scientific Scimed, Inc., Maple Grove, MN (US)

(21) Appl. No.: 18/454,442

(22) Filed: Aug. 23, 2023

Related U.S. Application Data
(60) Provisional application No. 63/400,980, filed on Aug. 25, 2022.

(51) Int. Cl. A61B 1/015; A61B 1/00

(52) U.S. Cl. CPC A61B 1/015; A61B 1/00137

(57) ABSTRACT

Methods and systems for coupling gas and water supply tubes to a container. An illustrative container and tube set may comprise a container configured to contain a fluid, the container having a bottom portion and a top portion, a water supply tube including a first end, a second end, and a first lumen, a gas supply tube including a first end, a second end, and a second lumen, and a weight coupled to the first end of the water supply tube and a first end of the gas supply tube. The first lumen may be in selective fluid communication with the bottom portion of the container and the second end of the water supply tube positioned external to the container. The second lumen may be in operative fluid communication with the container and the second end of the gas supply tube is positioned external to the container.

### CROSS-REFERENCE TO RELATED APPLICATIONS

This application claims the benefit of U.S. Provisional Patent Application Ser. No. 63/400,980 filed on Aug. 25, 2022, the disclosure of which is incorporated herein by reference.

### FIELD

This disclosure relates generally to medical fluid containers and methods, and particularly to a container and tube sets to supply fluid and/or gas to an endoscope.

### BACKGROUND

Conventionally, endoscope devices have been widely used for performing diagnostic and/or therapeutic treatments. During endoscopic procedures, physicians may use a combination of air, irrigation and lens wash as a means of flushing debris, cleaning optics, and insufflating the working lumen. To enable these features, the endoscope umbilicus is connected to a water bottle via a set of tubes. One of the tubes sends pressurized air from the processor to the water bottle. Another tube is a water tube that is suspended at the bottom of the bottle within the water. To ensure that the tube stays at the bottom of the water bottle, a weight may be coupled to the distal tip to keep the tube from floating to the top of the water surface. Additionally, at the top of the bottle, a cap is fitted with multiple features and parts to ensure that the preferred performance is achieved. It is with these considerations in mind that the improvements of the present disclosure may be useful.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary. Accordingly, while the disclosure is presented in terms of aspects or embodiments, it should be appreciated that individual aspects can be claimed separately or in combination with aspects and features of that embodiment or any other embodiment.

In a first example, a container and tube set arranged and configured to couple to an endoscope for use in an endoscopic procedure may comprise a container configured to contain a fluid, the container having a bottom portion and a top portion, a water supply tube including a first end, a second end, and a first lumen extending therethrough, wherein the first lumen is in selective fluid communication with the bottom portion of the container and the second end of the water supply tube is positioned external to the container, a gas supply tube including a first end, a second end, and a second lumen extending therethrough, wherein the second lumen is in operative fluid communication with the container and the second end of the gas supply tube is positioned external to the container, and a weight coupled to the first end of the water supply tube and a first end of the gas supply tube.

Alternatively or additionally to any of the examples above, in another example, the weight may comprise a housing having a housing lumen extending from a first end of the housing to a second end of the housing.

Alternatively or additionally to any of the examples above, in another example, the container and tube set may further comprise one or more apertures extending through a side wall of the housing, the one or more apertures positioned between the first and second ends of the housing.

Alternatively or additionally to any of the examples above, in another example, the housing lumen may have a cross-sectional dimension that incrementally decreases from the first end to the second end.

Alternatively or additionally to any of the examples above, in another example, the housing lumen may have a first cross-sectional dimension from the first end of the housing to a first intermediate location between the first end and the second end of the housing.

Alternatively or additionally to any of the examples above, in another example, the housing lumen may have a second cross-sectional dimension from the first intermediate location to a second intermediate location between the first end and the second end of the housing, the second cross-sectional dimension may be less than the first cross-sectional dimension.

Alternatively or additionally to any of the examples above, in another example, the housing lumen may have a third cross-sectional dimension from the second intermediate location to the second end, the third cross-sectional dimension may be less than the second cross-sectional dimension.

Alternatively or additionally to any of the examples above, in another example, a first transition in the cross-sectional dimension of the housing lumen may define a first ledge.

Alternatively or additionally to any of the examples above, in another example, the first end of the gas supply tube may be configured to abut the first ledge.

Alternatively or additionally to any of the examples above, in another example, the one or more apertures may be positioned between the first ledge and the second end of the housing.

Alternatively or additionally to any of the examples above, in another example, a second transition in the cross-sectional dimension of the housing lumen may define a second ledge.

Alternatively or additionally to any of the examples above, in another example, the first end of the water supply tube may be configured to abut the second ledge.

Alternatively or additionally to any of the examples above, in another example, a flow of gas through the second lumen may be configured to exit the one or more apertures.

Alternatively or additionally to any of the examples above, in another example, a flow of water may be configured to enter the first lumen through the second end of the housing upon pressurization of the container.

Alternatively or additionally to any of the examples above, in another example, the container and tube set may further comprise a one-way valve coupled to the one or more apertures.

Alternatively or additionally to any of the examples above, in another example, the one-way valve may comprise an umbrella valve.

Alternatively or additionally to any of the examples above, in another example, the one-way valve may be configured to allow a flow of gas to exit the housing into the container.

In another example, a container and tube set arranged and configured to couple to an endoscope for use in an endoscopic procedure may comprise a container configured to contain a fluid, the container having a bottom portion and a top portion, a water supply tube including a first end, a second end, and a first lumen extending therethrough, wherein the first lumen is in selective fluid communication with the bottom portion of the container and the second end of the water supply tube is positioned external to the container, a gas supply tube including a first end, a second end, and a second lumen extending therethrough, wherein the second lumen is in operative fluid communication with the container and the second end of the gas supply tube is positioned external to the container, and a weight coupled to the first end of the water supply tube and a first end of the gas supply tube. The weight may comprise a housing having a first housing lumen extending from a first end of the housing to a second end of the housing and a second housing lumen extending through a side wall of the housing.

Alternatively or additionally to any of the examples above, in another example, the container and tube set may further comprise one or more apertures formed in the second end of the housing.

Alternatively or additionally to any of the examples above, in another example, the first housing lumen may have a cross-sectional dimension that incrementally decreases from the first end to the second end.

Alternatively or additionally to any of the examples above, in another example, a first transition in the cross-sectional dimension of the housing lumen may define a first ledge.

Alternatively or additionally to any of the examples above, in another example, the first end of the gas supply tube may be configured to abut the first ledge.

Alternatively or additionally to any of the examples above, in another example, a first opening of the second housing lumen may be positioned between the first ledge and the second end of the housing.

Alternatively or additionally to any of the examples above, in another example, a second opening of the second housing lumen may be positioned adjacent to the second end of the housing.

Alternatively or additionally to any of the examples above, in another example, the first lumen of the water supply tube may be in fluid communication with the second housing lumen.

Alternatively or additionally to any of the examples above, in another example, the container and tube set may further comprise one or more apertures extending through the second end of the housing.

Alternatively or additionally to any of the examples above, in another example, the second lumen of the gas supply tube may be in fluid communication with the one or more apertures.

Alternatively or additionally to any of the examples above, in another example, the container and tube set may further comprise a one-way valve coupled to the one or more apertures.

Alternatively or additionally to any of the examples above, in another example, the one-way valve may comprise an umbrella valve.

Alternatively or additionally to any of the examples above, in another example, the one-way valve may be configured to allow a flow of gas to exit the housing into the container.

Alternatively or additionally to any of the examples above, in another example, the second housing lumen may extend at a non-orthogonal angle relative to the first housing lumen.

In another example, a container and tube set arranged and configured to couple to an endoscope for use in an endoscopic procedure may comprise a container configured to contain a fluid, the container having a bottom portion and a top portion, a water supply tube including a first end, a second end, and a first lumen extending therethrough, wherein the first lumen is in selective fluid communication with the bottom portion of the container and the second end of the water supply tube is positioned external to the container, a gas supply tube including a first end, a second end, and a second lumen extending therethrough, wherein the second lumen is in operative fluid communication with the container and the second end of the gas supply tube is positioned external to the container, and a weight coupled to the first end of the water supply tube and a first end of the gas supply tube. The weight may comprise a housing having a first housing lumen extending from a first end of the housing to a second end of the housing and one or more through holes radially spaced from the first housing lumen.

Alternatively or additionally to any of the examples above, in another example, the one or more through holes may be formed in the second end of the housing.

Alternatively or additionally to any of the examples above, in another example, the housing may have an inner cross-sectional dimension that incrementally decreases from the first end to the second end.

Alternatively or additionally to any of the examples above, in another example, a first transition in the inner cross-sectional dimension of the housing may define a first ledge.

Alternatively or additionally to any of the examples above, in another example, the first end of the gas supply tube may be configured to abut the first ledge.

Alternatively or additionally to any of the examples above, in another example, a second transition in the inner cross-sectional dimension of the housing may define a second ledge.

Alternatively or additionally to any of the examples above, in another example, the first end of the water supply tube may be configured to abut the second ledge.

Alternatively or additionally to any of the examples above, in another example, the first lumen of the water supply tube may be in fluid communication with the first housing lumen.

Alternatively or additionally to any of the examples above, in another example, the second lumen of the gas supply tube may be in fluid communication with the one or more through holes in the second end of the housing.

Alternatively or additionally to any of the examples above, in another example, the container and tube set may further comprise a one-way valve coupled to the one or more through holes.

Alternatively or additionally to any of the examples above, in another example, the one-way valve may comprise an umbrella valve.

Alternatively or additionally to any of the examples above, in another example, the one-way valve may be configured to allow a flow of gas to exit the housing into the container.

Alternatively or additionally to any of the examples above, in another example, the one-way valve may be configured to preclude a passage of water to into the housing.

In another example, a container arranged and configured to couple to an endoscope for use in an endoscopic procedure may comprise a flexible container configured to contain a fluid within a first receptacle thereof, the container having a bottom portion and a top portion, a water outlet positioned adjacent to the bottom portion of the container, a gas inlet, the gas inlet in fluid communication with a second receptacle of the container. The second receptacle may comprise a hydrophobic membrane.

Alternatively or additionally to any of the examples above, in another example, the hydrophobic membrane may be configured to allow gas to pass from the second receptacle to the first receptacle.

Alternatively or additionally to any of the examples above, in another example, the hydrophobic membrane may be configured to preclude a passage of water from the first receptacle to the second receptacle.

Alternatively or additionally to any of the examples above, in another example, the container may further comprise a water supply tube including a first end, a second end, and a first lumen extending therethrough, wherein the first lumen is in fluid communication with the first receptacle in the bottom portion of the container and the second end of the water supply tube is positioned external to the container and a gas supply tube including a first end, a second end, and a second lumen extending therethrough, wherein the second lumen is in operative fluid communication with the first receptacle and the second end of the gas supply tube is positioned external to the container.

Alternatively or additionally to any of the examples above, in another example, the container may further comprise a port positioned adjacent to the top portion of the container, wherein the port is configured to selectively fluidly couple the first receptacle of the container with an external water source and a removable cap selectively coupled to the port.

In another example, a container and tube set arranged and configured to couple to an endoscope for use in an endoscopic procedure may comprise a container configured to contain a fluid, the container having a bottom portion and a top portion, a water supply tube including a first end, a second end, and a first lumen extending therethrough, wherein the first lumen is in selective fluid communication with the bottom portion of the container and the second end of the water supply tube is positioned external to the container, and a gas supply tube including a first end, a second end, a second lumen extending therethrough, wherein the second lumen is in operative fluid communication with the container and the second end of the gas supply tube is positioned external to the container and wherein a first end of the gas supply tube is positioned inside the container, extends to the bottom portion of the container, and has a sidewall that is configured allow gas to pass from the second lumen into the container while water is inhibited from flowing from the container into the second lumen. The water supply tube may extend co-axially with the gas supply lumen and the first end of the water supply tube is generally aligned with a first end of the gas supply tube.

Alternatively or additionally to any of the examples above, in another example, an annular opening at the first end of the gas supply tube may be closed off.

Alternatively or additionally to any of the examples above, in another example, the container and tube set may further comprise a weight coupled the first end of the gas supply tube and/or the first end of the water supply tube.

Alternatively or additionally to any of the examples above, in another example, the sidewall may have a plurality of pin holes extending therethrough.

Alternatively or additionally to any of the examples above, in another example, the sidewall may be formed from an elastomer.

Alternatively or additionally to any of the examples above, in another example, the sidewall may be formed from a finely woven mesh.

Alternatively or additionally to any of the examples above, in another example, the sidewall may comprise a hydrophobic membrane.

In another example, a container and tube set arranged and configured to couple to an endoscope for use in an endoscopic procedure may comprise an outer chamber, an inner chamber disposed within the outer chamber and configured to contain a fluid, a water supply tube including a first end, a second end, and a first lumen extending therethrough, wherein the first lumen is in fluid communication with the inner chamber and the second end of the water supply tube is positioned external to the vessel, and a gas supply tube including a first end, a second end, and a second lumen extending therethrough, wherein the second lumen is in operative communication with the outer chamber and the second end of the gas supply tube is positioned external to the container.

Alternatively or additionally to any of the examples above, in another example, the outer chamber may be configured to compress the inner chamber to expel fluid from the inner chamber.

In another example, a container arranged and configured to couple to an endoscope for use in an endoscopic procedure may comprise a flexible container configured to contain a fluid within a first receptacle thereof, the container having a bottom portion and a top portion, a water outlet positioned adjacent to the bottom portion of the container, and a gas inlet, the gas inlet in fluid communication with an inner channel of the container. The inner channel may comprise a flow control mechanism disposed adjacent a second end thereof.

Alternatively or additionally to any of the examples above, in another example, the flow control mechanism may comprise a duckbill valve.

Alternatively or additionally to any of the examples above, in another example, the flow control mechanism may comprise an umbrella valve.

Alternatively or additionally to any of the examples above, in another example, the flow control mechanism may comprise a hydrophobic membrane.

Alternatively or additionally to any of the examples above, in another example, the flow control mechanism may be configured to preclude a passage of water from the first receptacle to the inner channel receptacle.

Alternatively or additionally to any of the examples above, in another example, the container may further comprise a water supply tube including a first end, a second end, and a first lumen extending therethrough, wherein the first lumen is in fluid communication with the first receptacle in the bottom portion of the container and the second end of the water supply tube is positioned external to the container and a gas supply tube including a first end, a second end, and a second lumen extending therethrough, wherein the second lumen is in operative fluid communication with the first receptacle and the second end of the gas supply tube is positioned external to the container.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various exemplary embodiments and together with the description serve to explain the principles of the present disclosure.

FIG. 1 depicts components of an endoscope;

FIG. 2 depicts components of an endoscope system with endoscope, light source, light source connector, water reservoir, and tubing assembly for air and lens wash fluid delivery;

FIG. 3A depicts an endoscope system with endoscope, light source, water reservoir, and tubing assembly for hybrid air, lens wash and irrigation fluid delivery, wherein the system is activated to deliver air to atmosphere;

FIG. 3B depicts the endoscope system of FIG. 3A, wherein the system is activated to deliver air to a patient through the patient end of the endoscope;

FIG. 3C depicts the endoscope system of FIG. 3A, wherein the system is activated to deliver lens wash fluid through the patient end of the endoscope;

FIG. 3D depicts the endoscope system of FIG. 3A, wherein the system is activated to deliver irrigation fluid through the patient end of the endoscope;

FIG. 4 depicts a hybrid endoscope system including a video processing unit, connector portion, peristaltic irrigation pump, water reservoir and top, coaxial gas and lens wash supply tubing, upstream and downstream irrigation supply tubing, and alternative gas supply tubing;

FIG. 5A depicts a perspective view of an illustrative distal tubing weight;

FIG. 5B depicts a perspective cross-sectional view of the illustrative distal tubing weight of FIG. 5A taken at line 5B-5B of FIG. 5A;

FIG. 5C depicts a perspective cross-sectional view of the illustrative distal tubing weight taken at line 5C-5C of FIG. 5A;

FIG. 5D depicts a cross-sectional view of the illustrative distal tubing weight of FIG. 5A assembled with a gas supply tube and a water supply tube;

FIG. 5E depicts a schematic view of the illustrative distal tubing weight of FIG. 5A assembled with a gas supply tube, a water supply tube, and a reservoir;

FIG. 6A depicts a perspective view of another illustrative distal tubing weight;

FIG. 6B depicts a perspective cross-sectional view of the illustrative distal tubing weight of FIG. 6A taken at line 6B-6B of FIG. 6A;

FIG. 6C depicts a top view of the illustrative distal tubing weight of FIG. 6A;

FIG. 6D depicts a cross-sectional view of the illustrative distal tubing weight of FIG. 6A assembled with a gas supply tube and a water supply tube;

FIG. 7A depicts a top perspective view of another illustrative distal tubing weight;

FIG. 7B depicts a bottom perspective of the illustrative distal tubing weight of FIG. 7A;

FIG. 7C depicts a top view of the illustrative distal tubing weight of FIG. 7A;

FIG. 7D depicts a cross-sectional view of the illustrative distal tubing weight of FIG. 7A assembled with a gas supply tube and a water supply tube;

FIG. 8A depicts a top perspective view of another illustrative distal tubing weight;

FIG. 8B depicts a perspective cross-sectional view of the illustrative distal tubing weight of FIG. 8A taken at line 8B-8B of FIG. 8A;

FIG. 8C depicts a top view of the illustrative distal tubing weight of FIG. 8A;

FIG. 8D depicts a bottom view of the illustrative distal tubing weight of FIG. 8A;

FIG. 8E is a cross-sectional view of the illustrative distal tubing weight of FIG. 8A assembled with a gas supply tube and a water supply tube;

FIG. 9A depicts a top perspective view of another illustrative distal tubing weight;

FIG. 9B depicts a perspective cross-sectional view of the illustrative distal tubing weight of FIG. 9A taken at line 9B-9B of FIG. 9A;

FIG. 9C depicts a top view of the illustrative distal tubing weight of FIG. 9A;

FIG. 9D depicts a bottom view of the illustrative distal tubing weight of FIG. 9A;

FIG. 9E is a cross-sectional view of the illustrative distal tubing weight of FIG. 9A assembled with a gas supply tube and a water supply tube;

FIG. 10 depicts a side view of an illustrative refillable fluid reservoir;

FIG. 11A depicts a side view of another illustrative refillable fluid reservoir and tube set;

FIG. 11B depicts an enlarged view of region B of FIG. 11A;

FIG. 12 depicts another illustrative reservoir for use with an endoscope system;

FIG. 13A depicts a cross-sectional side view of an illustrative fluid reservoir in a first configuration; and

FIG. 13B depicts a schematic side view of the illustrative reservoir of FIG. 13A in a second configuration.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an exemplary medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, the same or similar reference numbers will be used through the drawings to refer to the same or like parts.

The term “distal” refers to a portion farthest away from a user when introducing a device into a patient. By contrast, the term “proximal” refers to a portion closest to the user when placing the device into the patient. As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” Further, as used herein, the terms “about,” “approximately” and “substantially” indicate a range of values within +/−10% of a stated or implied value. Additionally, terms that indicate the geometric shape of a component/surface refer to exact and approximate shapes.

Embodiments of the present disclosure are described with specific reference to a bottle (e.g., container, reservoir, or the like) and tube assembly or set. It should be appreciated that such embodiments may be used to supply fluid and/or gas to an endoscope, for a variety of different purposes, including, for example to facilitate insufflation of a patient, lens washing, and/or to irrigate a working channel to aid in flushing/suctioning debris during an endoscopic procedure.

Although the present disclosure includes descriptions of a container and tube set suitable for use with an endoscope system to supply fluid and/or gas to an endoscope, the devices, systems, and methods herein could be implemented in other medical systems requiring fluid and/or gas delivery, and for various other purposes.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

Conventionally, endoscope devices have been widely used for performing diagnostic and/or therapeutic treatments. During endoscopic procedures, physicians may use a combination of air, irrigation and lens wash as a means of flushing debris, cleaning optics, and insufflating the working lumen. To enable these features, the endoscope umbilicus is connected to a water bottle via a set of tubes. One of the tubes sends pressurized air from the processor to the water bottle. Another tube is a water tube that is suspended at the bottom of the bottle within the water. To ensure that the tube stays at the bottom of the water bottle, a weight may be coupled to the distal tip to keep the tube from floating to the top of the water surface. Additionally, at the top of the bottle, a cap is fitted with multiple features and parts to ensure that the preferred performance is achieved. Disclosed herein are containers and tube sets which combing multiple parts and features into one part which may reduce the number of parts needed to achieve the same performance.

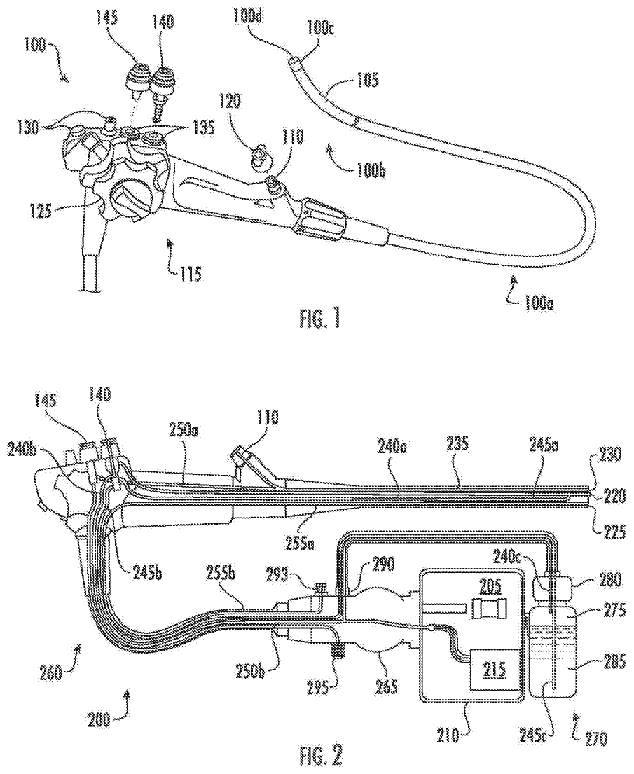

With reference to FIGS. 1-2, an exemplary endoscope 100 and system 200 are depicted that may comprise an elongated shaft 100a that is inserted into a patient. A light source 205 feeds illumination light to a distal portion 100b of the endoscope 100, which may house an imager (e.g., CCD or CMOS imager) (not shown). The light source 205 (e.g., lamp) is housed in a video processing unit 210 that processes signals that are input from the imager and outputs processed video signals to a video monitor (not shown) for viewing. The video processing unit 210 also serves as a component of an air/water feed circuit by housing a pressurizing pump 215, such as an air feed pump, in the unit.

The endoscope shaft 100a may include a distal tip 100c provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 extends along the shaft 100a to a proximal channel opening 110 positioned distal to an operating handle 115 of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the bendable flexible portion 105 (e.g., one knob controls up-down steering and another knob control for left-right steering). A plurality of video switches 130 for remotely operating the video processing unit 210 may be arranged on a proximal end side of the handle 115. In addition, the handle 115 is provided with dual valve wells 135. One of the valve wells 135 may receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220 (FIG. 2). The other valve well 135 receives a suction valve 145 for operating a suction operation. A suction supply line 250a runs distally from the suction valve 145 along the shaft 100a to a junction point in fluid communication with the working channel 235 of the endoscope 100.

The operating handle 115 is electrically and fluidly connected to the video processing unit 210, via a flexible umbilical 260 and connector portion 265 extending therebetween. The flexible umbilical 260 has a gas (e.g., air or CO₂) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the air pump 215 to the gas feed line 240b in the umbilical 260.

A water reservoir or container 270 (e.g., water bottle) is fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir to a detachable gas/lens wash connection 290 on the outside of the connector portion 265. The detachable gas/lens wash connection 290 may be detachable from the connector portion 265 and/or the gas supply tubing 240c. The gas feed line 240b from the umbilical 260 branches in the connector portion 265 to fluidly communicate with the gas supply tubing 240c at the detachable gas/lens wash connection 290, as well as the air pump 215. A length of lens wash tubing 245c, with one end positioned at the bottom of the reservoir 270, passes through the top 280 of the reservoir 270 to the same detachable connection 290 as the gas supply tubing 240c on the connector portion 265. In other embodiments, the connections may be separate and/or separated from each other. The connector portion 265 also has a detachable irrigation connection 293 for irrigation supply tubing (not shown) running from a source of irrigation water (not shown) to the irrigation feed line 255b in the umbilical 260. The detachable irrigation connection 293 may be detachable from the connector portion 265 and/or the irrigation supply tubing (not shown). In some embodiments, irrigation water is supplied via a pump (e.g., peristaltic pump) from a water source independent (not shown) from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100. The detachable suction connection 295 may be detachable from the connector portion 265 and/or the suction feed line 250b and/or the vacuum source.

The gas feed line 240b and lens wash feed line 245b are fluidly connected to the valve well 135 for the gas/water valve 140 and configured such that operation of the gas/water valve in the well controls supply of gas or lens wash to the distal tip 100c of the endoscope 100. The suction feed line 250b is fluidly connected to the valve well 135 for the suction valve 145 and configured such that operation of the suction valve in the well controls suction applied to the working channel 235 of the endoscope 100.

Referring to FIG. 2, an exemplary operation of an endoscopic system 200, including an endoscope such as endoscope 100 above, is explained. Air from the air pump 215 in the video processing unit 210 is flowed through the connection portion 265 and branched to the gas/water valve 140 on the operating handle 115 through the gas feed line 240b in the umbilical 260, as well as through the gas supply tubing 240c to the water reservoir 270 via the connection 290 on the connector portion 265. When the gas/water valve 140 is in a neutral position, without the user's finger on the valve, air is allowed to flow out of the valve to atmosphere. In a first position, the user's finger is used to block the vent to atmosphere. Gas is allowed to flow from the valve 140 down the gas supply line 240a and out the distal tip 100c of the endoscope 100 in order to, for example, insufflate the treatment area of the patient. When the gas/water valve 140 is pressed downward to a second position, gas is blocked from exiting the valve, allowing pressure of the air passing from the air pump 215 to rise in the water reservoir 270. Pressurizing the water source forces water out of the lens wash tubing 245c, through the connector portion 265, umbilical 260, through the gas/water valve 140 and down the lens wash supply line 245a, converging with the gas supply line 240a prior to exiting the distal tip 100c of the endoscope 100 via the gas/lens wash nozzle 220. Air pump pressure may be calibrated to provide lens wash water at a relatively low flow rate compared to the supply of irrigation water.

The volume of the flow rate of the lens wash is governed by gas pressure in the water reservoir 270. When gas pressure begins to drop in the water reservoir 270, as water is pushed out of the reservoir 270 through the lens wash tubing 245c, the air pump 215 replaces lost air supply in the reservoir 270 to maintain a substantially constant pressure, which in turn provides for a substantially constant lens wash flow rate. In some embodiments, a filter (not shown) may be placed in the path of the gas supply tubing 240c to filter-out undesired contaminants or particulates from passing into the water reservoir 270. In some embodiments, outflow check valves or other one-way valve configurations (not shown) may be placed in the path of the lens wash supply tubing to help prevent water from back-flowing into the reservoir 270 after the water has passed the valve.

A relatively higher flow rate of irrigation water is typically required compared to lens wash, since a primary use is to clear the treatment area in the patient of debris that obstructs the user's field of view. Irrigation is typically achieved with the use of a pump (e.g., peristaltic pump), as described. In embodiments with an independent water source for irrigation, tubing placed in the bottom of a water source is passed through the top of the water source and threaded through the head on the upstream side of the pump. Tubing on the downstream side of the pump is connected to the irrigation feed line 255b in the umbilical 260 and the irrigation supply line 255a endoscope 100 via the irrigation connection 293 on the connector portion 265. When irrigation water is required, fluid is pumped from the water source by operating the irrigation pump, such as by depressing a footswitch (not shown), and flows through the irrigation connection 293, through the irrigation feed line 255b in the umbilical, and down the irrigation supply line in the shaft 100a of the endoscope to the distal tip 100c. In order to equalize the pressure in the water source as water is pumped out of the irrigation supply tubing, an air vent (not shown) may be included in the top 280 of the water reservoir 270. The vent allows atmospheric air into the water source preventing negative pressure build-up in the water source, which could create a vacuum that suctions undesired matter from the patient back through the endoscope toward the water source. In some embodiments, outflow check valves or other one-way valve configurations (not shown), similar to the lens wash tubing 245c, may be placed in the path of the irrigation supply tubing to help prevent back-flow into the reservoir after water has passed the valve.

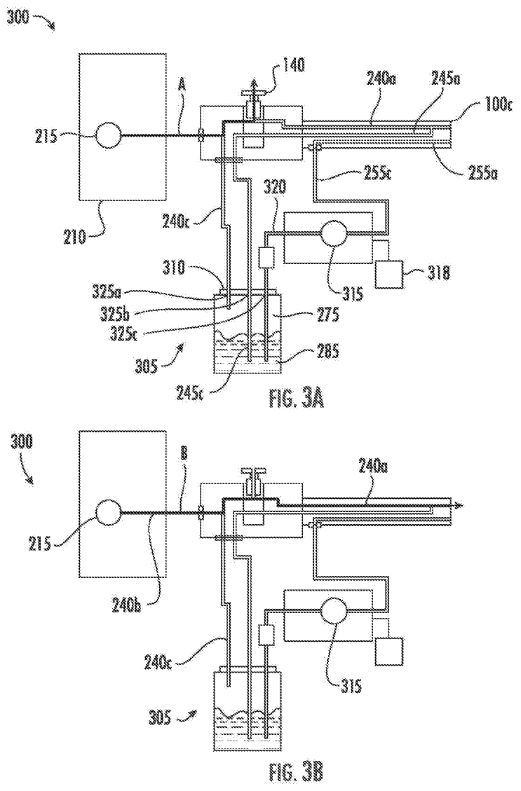

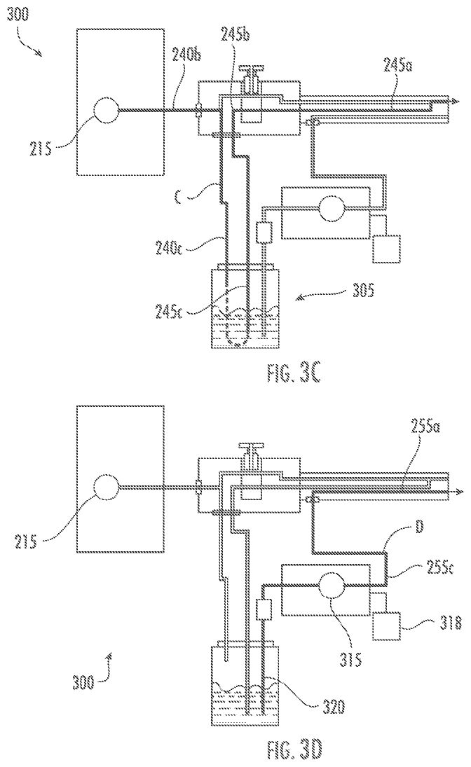

FIGS. 3A-3D are schematic drawings illustrating the operation of an embodiment of a hybrid system 300 where the supply tubing for irrigation and lens wash are connected to and drawn from a single water reservoir. It is contemplated that fluids other than water may be used, such as, but not limited to saline. The hybrid system 300 includes the single water reservoir 305, a cap 310 for the reservoir, gas supply tubing 240c, lens wash supply tubing 245c, irrigation pump 315 with foot switch 318, upstream irrigation tubing 320 and downstream irrigation supply tubing 255c. The cap 310 may be configured to attach in a seal-tight manner to the water reservoir 305 by a typically threaded arrangement. The cap 310 may include a gasket to seal the cap 310 to the reservoir 305. The gasket can be an O-ring, flange, collar, and/or the like and can be formed of any suitable material. A number of through-openings (325a, 325b, 325c) in the cap 310 are provided to receive, respectively, the gas supply tubing 240c, lens wash supply tubing 245c, and upstream irrigation supply tubing 320. In FIGS. 3A-3D, the system depicted includes separate tubing for gas supply, lens wash, and irrigation.

In other embodiments, the gas supply tubing 240c and lens wash tubing 245c may be combined in a coaxial arrangement. Some illustrative coaxial arrangements are described in commonly assigned U.S. patent application Ser. No. 17/558,239, titled INTEGRATED CONTAINER AND TUBE SET FOR FLUID DELIVERY WITH AN ENDOSCOPE and U.S. patent application Ser. No. 17/558,256, titled TUBING ASSEMBLIES AND METHODS FOR FLUID DELIVERY, the disclosures of which are hereby incorporated by reference. For example, the gas supply tubing may define a lumen that is sufficiently large in diameter to encompass a smaller diameter lens wash tubing, coaxially received within the gas supply tubing, as well as provide air to the water source in an annular space surrounding the lens wash tubing to pressurize the water reservoir (see, e.g., gas and lens wash supply tubing 240c, 245c). The lens wash supply tubing may be configured to exit the lumen defined by the coaxial gas supply tubing in any suitable sealed manner, such as, for example, an aperture, fitting, collar, and/or the like, for the purpose of transitioning from the coaxial arrangement to a side-by-side arrangement at the detachable gas/lens wash connection to the endoscope connector portion (e.g., connector portion 265 of FIG. 2).

In various embodiments, different configurations of valving (not shown) may be incorporated into various embodiments disclosed hereby, including the tubing of the system 200, 300. For example, an in-flow check valve can be disposed in the path of the gas supply tubing 240c to help prevent backflow into the air pump 215. In this manner, pressure building within the water reservoir 305 creates a pressure difference between the water source and the gas supply tubing 240c helping to maintain a positive pressure in the water source even when large amounts of water may be removed from the water source during the irrigation function. This arrangement compensates for any time lag in air being delivered from the air pump 215 to the water reservoir 305, which might otherwise cause a negative pressure vacuum in the water reservoir. Similarly, an out-flow check valve, such as the one-way valve with inlet/outlets and valve insert, may be incorporated in the lens wash supply tubing 240c, upstream irrigation supply tubing 320, and/or downstream irrigation supply tubing 255c to help prevent backflow of water from either or both of the lens wash and irrigation tubing in the event of a negative pressure situation, as described.

More generally, in many embodiments, a check valve may refer to any type of configuration for fluid to flow only in one direction in a passive manner. For example, a check valve may include, or refer to, one or more of a ball check valve, a diaphragm check valve, a swing check valve, a tilting disc check valve, a flapper valve, a stop-check valve, a lift-check valve, an in-line check valve, a duckbill valve, a pneumatic non-return valve, a reed valve, a flow check. Accordingly, a check valve as used herein is meant to be separate and distinct from an active valve that is operated in a binary manner as an on/off valve or switch to allowed flow to be turned on or allow flow to be turned off (e.g., a stop cock valve, solenoid valve, peristaltic pump).

During operation of the system of FIGS. 3A-3D, a flow of water for irrigation may be achieved by operating the irrigation pump 315. A flow of water for lens wash may be achieved by depressing the gas/water valve 140 on the operating handle 115 of the endoscope 100. These functions may be performed independent of one another or simultaneously. When operating lens wash and irrigation at the same time, as fluid is removed from the water reservoir 305, the pressure in the system may be controlled to maintain the lens wash supply tubing 240c at substantially the pressure necessary to accomplish a lower flow rate lens wash, while compensating for reduced pressure in the water reservoir 305 due to supplying a high flow rate irrigation. When pressure is reduced in the water reservoir by use of the lens wash function, the irrigation function, or both functions simultaneously, the reduced pressure may be compensated for by the air pump 215 via the gas supply tubing 240c.

The schematic set-up in FIGS. 3A-3D has been highlighted to show the different flow paths possible with the hybrid system 300 having supply tubing for irrigation 320 and lens wash 240c connected to and drawn from the single water reservoir 305. As shown in FIG. 3A, the endoscope 100 is in a neutral state with the gas/water valve 140 in an open position. The neutral state delivers neither gas, nor lens wash, to the distal tip of the endoscope. Rather gas (pressure) is delivered along path A from the pressurizing air pump 215 and vented through the gas feed line 240b in the umbilical 260 via the connector portion 265 and through the gas/water valve to atmosphere. Since the system is open at the vent hole in the gas/water valve 140, there is no build up to pressurize the water reservoir 305 and consequently no water is pushed through the lens wash supply tubing 240c.

As shown in FIG. 3B, the endoscope 100 is in a gas delivery state with the gas/water valve 140 in a first position. When gas is called for at the distal tip 100c, for example, to clean the end face 100d of the distal tip or insufflate the patient body in the treatment area, the user closes off the vent hole in the gas/water valve 140 with a thumb, finger, or the like (first position). In this state, gas (pressure) is delivered along path B from the air pump 215 and flowed through the gas feed line 240b in the umbilical 260 via the connector portion 265. The gas continues through the gas/water valve 140 to the gas supply line 240a in the endoscope shaft 100a and out the gas/lens wash nozzle 220 at the distal tip 100c. There is no build up to pressurize the water reservoir since the system is open at the gas/lens water nozzle 220, and consequently no water is pushed through the lens wash supply tubing 240c.

As shown in FIG. 3C, the endoscope 100 is in a lens wash delivery state with the gas/water valve 140 in a second position. When lens wash is called for at the distal tip 100c, for example, to clean the end face 100d of the distal tip 100c, the user, keeping the vent hole in the air/water valve closed off, depresses the valve 140 to its furthest point in the valve well 135. The second position blocks off the gas supply to both atmosphere and the gas supply line 240a in the endoscope, and opens up the gas/water valve 140 to allow lens wash water to pass through to the lens wash supply line 245a in the endoscope shaft 100a and out the gas/lens wash nozzle 220 at the distal tip 100c. In this state, gas (pressure) is delivered along path C from the air pump 215, through the branched line in the connector portion 265 and out of the gas supply tubing 240c to the water reservoir 305. The gas (pressure) pressurizes the surface of the remaining water 285 in the reservoir 305 and pushes water up the lens wash supply tube 245c to the connector portion 265. The pressurized lens wash water is pushed further through the lens wash feed line 245b in the umbilical 260 and through the gas/water valve 140. Since the system 300 is closed, gas pressure is allowed to build and maintain a calibrated pressure level in the water reservoir 305, rather than venting to atmosphere or being delivered to the patient. This pressure, along with the endoscope feed and supply lines and external tubing, translates to a certain range of flow rate of the lens wash.

As shown in FIG. 3D, the endoscope 100 is in an irrigation delivery state. This may be performed at the same or a different time from the delivery of gas and/or lens wash. When irrigation is called for at the distal tip 100c, for example, if visibility in the treatment area is poor or blocked by debris, or the like, the user activates the irrigation pump 315 (e.g., by depressing foot switch 318) to delivery water along path D. With the pump 315 activated, water is sucked out of the water reservoir 305 through the upstream irrigation supply tubing 320 and pumped along the downstream irrigation supply tubing 255c to the connector portion 265. The irrigation pump head pressure pushes the irrigation water further through the irrigation feed line 255b in the umbilical 260, through the irrigation supply line 255a in the endoscope shaft 100a, and out the irrigation opening 225 at the distal tip 100c. The irrigation pump pressure may be calibrated, along with the endoscope irrigation feed and supply lines and external tubing, to deliver a certain range of flow rate of the irrigation fluid.

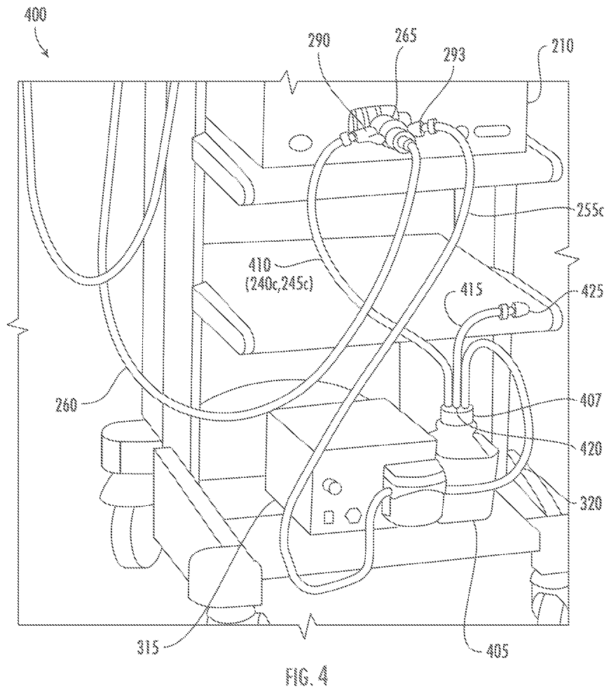

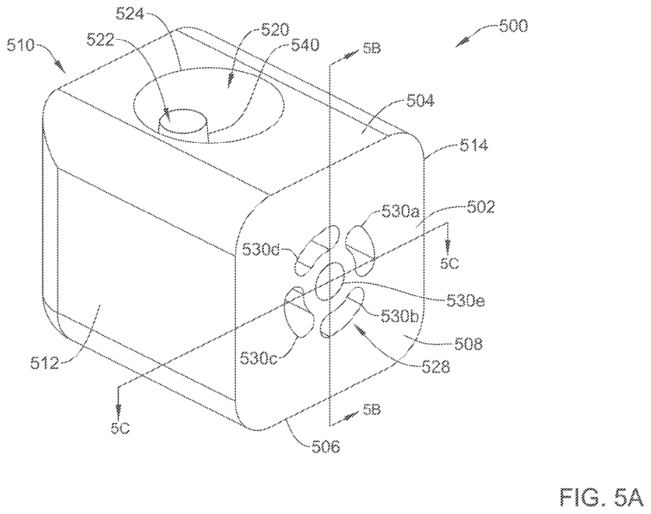

FIG. 4 is a schematic drawing illustrating a further embodiment of a hybrid system 400 including a video processing unit 210, connector portion 265, peristaltic irrigation pump 315, water reservoir 405 and top 407, coaxial gas and lens wash supply tubing 410, upstream and downstream irrigation supply tubing 320, 255c, respectively, and alternative gas (e.g., CO₂) supply tubing 415. A length of the alternative gas supply tubing 415 passes from one end positioned in the gas gap 275 (see FIG. 2) between the top 407 of the water reservoir 405 and the remaining water 285 in the reservoir through an additional opening 420 in the top of the reservoir to a detachable connection 425 for a source of the alternative gas supply (e.g., CO₂ hospital house gas source). When the alternative gas supply is desired, such as CO₂ gas, the air pump 215 on the video processing unit 210 may be turned off and CO₂ gas, rather than air, is thereby flowed to the water reservoir 405 pressurizing the water surface. Generally, the flow of CO₂ through the endoscope 100 is similar to the flow of air. In the neutral state, CO₂ gas flows backward up the gas supply tubing 240c to the connector portion 265, up the gas feed line 240b, and is vented through the gas/water valve 140 to atmosphere. In the first position, the user closes off the vent hole in the gas/water valve 140, and the CO₂ gas is flowed through the gas/water valve to the gas supply line 240a in the endoscope shaft 100a and out the gas/lens wash nozzle 220 at the distal tip 100c. In the second position, the user depresses the valve 140 to the bottom of the valve well 135, keeping the vent hole in the gas/water valve closed off. The second position blocks the CO₂ gas supply to both atmosphere and the gas supply line 240a in the endoscope 100, and opens up the gas/water valve 140 to allow lens wash water to pass through to the lens wash supply line 245a in the endoscope shaft 100a and out the gas/lens wash nozzle 220 at the distal tip 100c. Gas (pressure) in the reservoir 405 is maintained by delivery gas through alternative gas (e.g., CO₂) supply tubing 415. The irrigation function may be accomplished in a similar manner as the operation described above with respect to FIG. 3D.

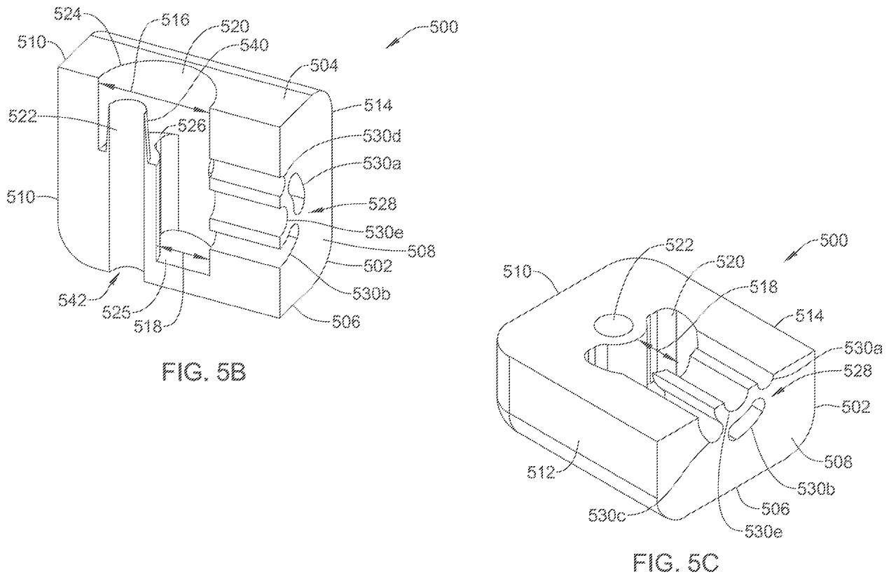

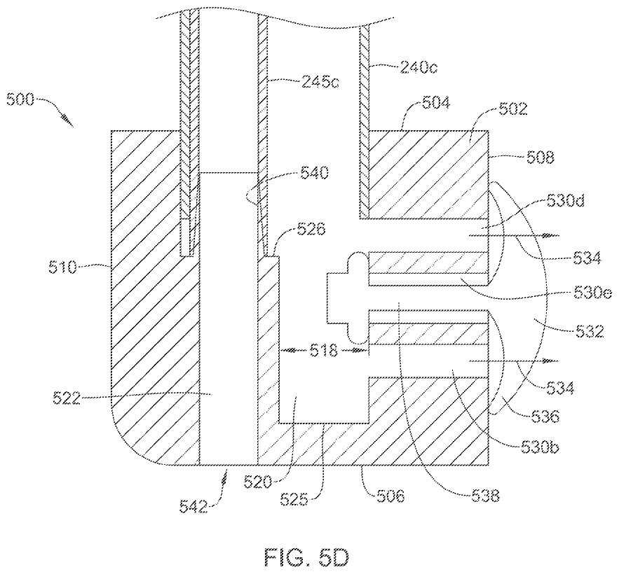

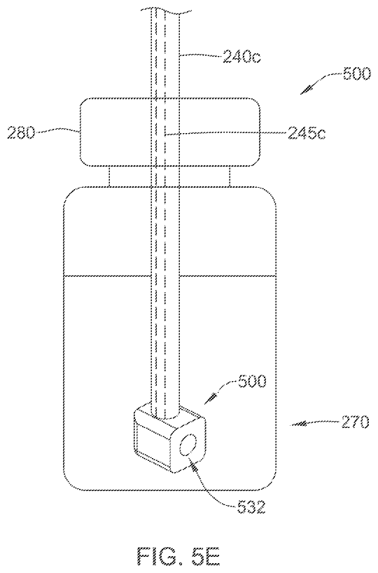

As described above, it may be desirable to reduce the number of parts in the system 200 while achieving the same performance. FIG. 5A depicts a perspective view of an illustrative distal tubing weight 500 for use with the gas supply tubing 240c, the lens wash tubing 245c, and the reservoirs 270, 305, 405. FIG. 5B depicts a perspective cross-sectional view of the illustrative distal tubing weight 500 taken at line 5B-5B of FIG. 5A. FIG. 5C depicts a perspective cross-sectional view of the illustrative distal tubing weight 500 taken at line 5C-5C of FIG. 5A. FIG. 5D depicts a cross-sectional view of the illustrative distal tubing weight 500 assembled with a gas supply tube 240c and a water supply tube 245c. FIG. 5E depicts a schematic view of the illustrative distal tubing weight 500 assembled with a gas supply tube 240c, a water supply tube 245c, and a reservoir 270. The distal tubing weight 500 may be configured to house the gas supply tube 240c and the water supply tube 245c within the reservoir 270, 305, 405 in a configuration that reduces the complexity of the water bottle cap or top 280, 407 while also maintaining the gas supply tube 240c and water supply tube 245c in a desired configuration.

The distal tubing weight 500 includes a housing 502 extending from a first, or proximal, end 504 to a second, or distal, end 506. In some cases, the first end 504 may be considered a top of the housing 502 while the second end 506 may be considered a bottom of the housing 502. The illustrative housing 502 includes a front side 508, a back side 510, and at least a first side 512, and a second opposing side 514. The first and second sides 512, 514 may each extend from or between the front 508 to the back 510. The first and second ends 504, 506 may extend from or between the first and second sides 512, 514. The use of the terms “front”, “back”, “first”, “second”, “top”, and “bottom” are not intended to limit the distal tubing weight 500 to a particular orientation, but rather facilitate discussion of relative orientation. Further, the housing 502 is not limited to a rectangular or generally rectangular structure. Other shapes may be used for the housing 502, as desired, including a cylindrical or pyramidal structure, among others.

The housing 502 may define a first housing lumen 520 extending distally from the first end 504 to a point proximal to the second end 506. The first housing lumen 520 may define an opening 524 in the first end 504 of the housing 502 and terminate at a second end 525 proximal to the second end 506 of the housing 502. The first housing lumen 520 may vary in cross-sectional shape and/or dimension along a length thereof. For example, the first housing lumen 520 may have a first cross-sectional shape having a first cross-sectional dimension 516 adjacent to the first end 504 of the housing 502 and a second cross-sectional shape having a second cross-sectional dimension 518 adjacent to the second end 506 of the housing 502. In the illustrated embodiments, the first cross-sectional shape of the first housing lumen 520 may be generally circular while the second cross-sectional shape of the first housing lumen 520 may have a generally “C” shape or crescent shape. However, the first cross-sectional shape and/or the second cross-sectional shape may take other cross-sectional shapes, as desired. It is further contemplated that in some embodiments, the first cross-sectional shape and the second cross-sectional shape may be same general shape. The second cross-sectional dimension 518 may be less than the first cross-sectional dimension 516. The cross-sectional dimension 516, 518 (and/or shape) of the first housing lumen 520 may change in an abrupt or step-wise manner to define a first shoulder or ledge 526.

The housing 502 may further define an air outlet 528 extending through a side wall thereof. While the air outlet 528 is illustrated as extending through the front side wall 508, the air outlet 528 may extend through any side wall 508, 510, 512, 514 desired. In yet other embodiments, the air outlet 528 may extend through the second end 506 of the housing 502. The air outlet 528 may include a plurality of apertures 530a-e. While the first air outlet 528 is shown and described as having five apertures 530a-e, the first air outlet 528 may have fewer than five or more than five apertures, as desired. The air outlet 528 is in fluid communication with the first housing lumen 520 and is configured to be in fluid communication with the lumen of the gas supply tube 240c via the first housing lumen 520.

A one-way valve 532 (FIG. 5D) may positioned in or adjacent to the first air outlet 528. In some examples, the one-way valve 532 may be a flap valve although other one-way valves, including those described elsewhere herein, may be used, as desired. The one-way valve 532 may be configured to allow air to move from the first housing lumen 520 of the housing 502 and exit the via the apertures 530a-d as shown at arrows 534. For example, air or gas flowing through the first housing lumen 520 may deflect the flaps 536 away from the housing 502. However, the one-way valve 532 may prevent air from moving in the opposite direction. This may allow air to enter and pressurize the reservoir. The one-way valve 532 may also prevent water from entering the first housing lumen 520 of the housing 502. The one-way valve 532 may be coupled to the first air outlet 528 using a number of techniques including, but not limited to, glue, adhesives, sonic welding, ultrasound welding, etc. In some cases, a central post 538 of the one-way valve 532 may extend through the central aperture 530e to secure the one-way valve 532 to the housing 502, for example, by a snap fit or friction fit.

The gas supply tube 240c may extend into the first housing lumen 520 of housing 502, as shown in FIG. 5D. In some embodiments, at least a portion of the first end of the gas supply tube 240c may abut the first ledge 526. However, this is not required. In some embodiments, the first end of the gas supply tube 240c may be proximal to the first shoulder 526. The gas supply tube 240c may be secured to the housing 502 using a number of techniques, including, but not limited to, friction fits, snap fits, glue, adhesive, etc. As described above, air from the air pump 215 (or gas from an alternative source) may flow through the connection portion 265 and to the reservoir 270 (depending on the position of the gas/water valve 140). As air exits the lumen of the gas supply tube 240c, the air enters the first housing lumen 520 and exits the housing 502 via the air outlet 528. The one-way valve 532 allows air to enter the reservoir 270, 305, 405 to pressurize it but does not allow for the air to re-enter the housing 502 and/or the gas supply tube 240c. It is contemplated that the first end of the gas supply tube 240c and/or the air outlet 528 may be positioned proximal to a water inlet 542 so that air may enter the housing 502 and exit into the reservoir but not make its way up the water supply tube 245c.

The housing 502 may further include a second housing lumen 522 which may extend distally from a point distal to the first end 504 to the second end 506. In some cases, a portion of the second housing lumen 522 may be defined by a tubular member 540 extending proximally from the first shoulder 526. However, this is not required. In some embodiments, the second housing lumen 522 may begin at the first shoulder 526 and extend distally therefrom. The tubular member 540 may increase in outer diameter in the distal direction. While this is not required, the increasing diameter may facilitate coupling of the water supply tube 245c with the tubular member 540.

The second housing lumen 522 may be configured to be in fluid communication with the water supply tube 245c. In some embodiments, the water supply tube 245c may be disposed over the tubular member 540 to fluidly couple the lumen of the water supply tube 245c with the second housing lumen 522, as shown in FIG. 5D. In some embodiments, at least a portion of the first end of the water supply tube 245c may abut the first shoulder 526. However, this is not required. In other embodiments, the first end of the water supply tube 245c may be inserted into the second housing lumen 522. When the water supply tube 245c is fluidly coupled with the second housing lumen 522, the first housing lumen 520 and the second housing lumen 522 are fluidly isolated from one another. The water supply tube 245c may extend through a lumen of the gas supply tube 240c such that only a single opening in the cap 280, 407 is necessary. The water supply tube 245c may extend through the gas supply tube 240c such that the longitudinal axis of the water supply tube 245c is laterally offset from the longitudinal axis of the gas supply tube 240c. In other examples, the water supply tube 245c and the gas supply tube 240c may extend co-axially. When the reservoir 270, 305, 405 is pressurized, water may enter the housing 502 via a water inlet 542 at a distal end of the second housing lumen 522. Water may then flow through the second housing lumen 522 proximally and into the lumen of the water supply tube 245c to provide lens wash capabilities.

The housing 502 may be formed from a material that has a density greater than water. This may allow the housing 502 to act as a weight to keep the gas supply tube 240c and the water supply tube 245c from floating to the top of the reservoir 270, 305, 405. The housing 502 may begin a substantially solid member with the lumens 520, 522 and apertures 530a-e separately formed. For example, the lumens 520, 522 and apertures 530a-e may be machined into a substantially solid housing. In other examples, the housing 502 may be molded as a single monolithic structure to include the lumens 520, 522 and apertures 530a-e.

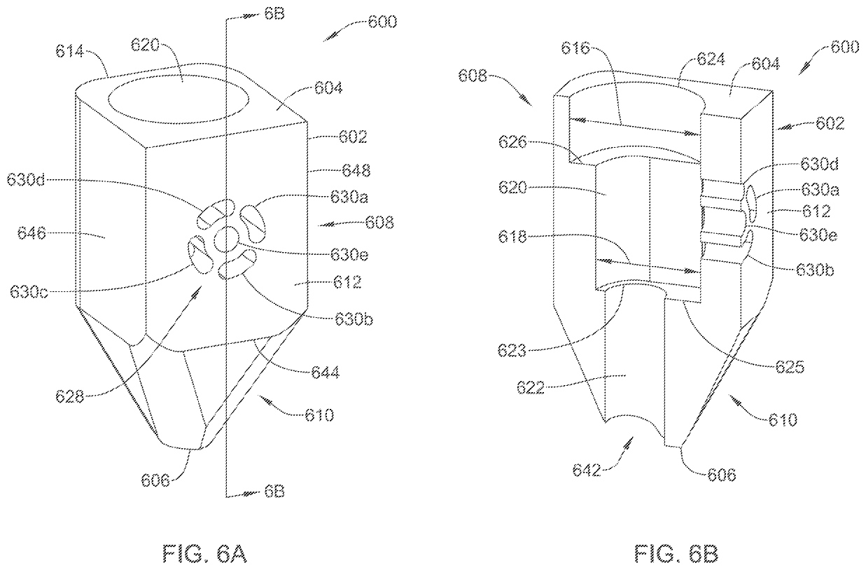

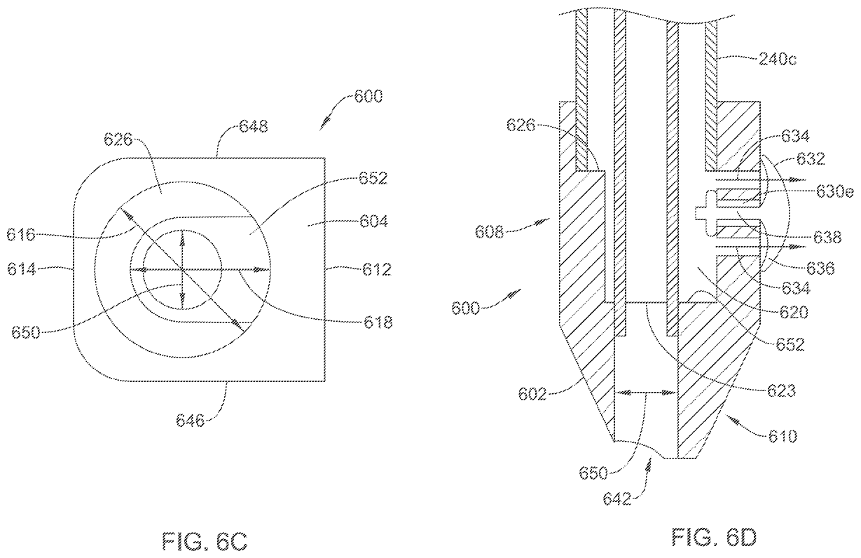

FIG. 6A depicts a perspective view of another illustrative distal tubing weight 600 for use with the gas supply tubing 240c, the lens wash tubing 245c, and the reservoirs 270, 305, 405. FIG. 6B depicts a perspective cross-sectional view of the illustrative distal tubing weight 600 taken at line 6B-6B of FIG. 6A. FIG. 6C depicts a top view of the illustrative distal tubing weight 600 of FIG. 6A. FIG. 6D depicts a cross-sectional view of the illustrative distal tubing weight 600 assembled with a gas supply tube 240c and a water supply tube 245c. The distal tubing weight 600 may be configured to house the gas supply tube 240c and the water supply tube 245c within the reservoir 270, 305, 405 in a configuration that reduces the complexity of the water bottle cap or top 280, 407 while also maintaining the gas supply tube 240c and water supply tube 245c in a desired configuration.

The distal tubing weight 600 includes a housing 602 extending from a first, or proximal, end 604 to a second, or distal, end 606. In some cases, the first end 604 may be considered a top of the housing 602 while the second end 606 may be considered a bottom of the housing 602. The illustrative housing 602 may include a first portion 608 having a generally rectangular prism shape and a second portion 610 having a generally truncated pyramidal shape. However, the housing 602 is not limited to a rectangular or generally rectangular structure or a pyramidal structure. Other shapes or combinations of shapes may be used for the housing 602, as desired, including a cylindrical structure, among others. The first portion 608 includes a front side 612, a back side 614, and at least a first side 646, and a second opposing side 648. The first and second sides 646, 648 may each extend from or between the front 612 to the back 614. The first end 604 may extend from or between the first and second sides 646, 648. The second portion 610 may extend distally from a second end 644 of the first portion 608 and may include a plurality of faces. The use of the terms “front”, “back”, “first”, “second”, “top”, and “bottom” are not intended to limit the distal tubing weight 600 to a particular orientation, but rather facilitate discussion of relative orientation.

The housing 602 may define a first housing lumen 620 extending distally from the first end 604 to a point proximal to the second end 606. The first housing lumen 620 may define an opening 624 in the first end 604 of the housing 602 and terminate at a second end 625 proximal to the second end 606 of the housing 602. The first housing lumen 620 may vary in cross-sectional shape and/or dimension along a length thereof. For example, the first housing lumen 620 may have a first cross-sectional shape having a first cross-sectional dimension 616 adjacent to the first end 604 of the housing 602 and a second cross-sectional shape having a second cross-sectional dimension 618 adjacent to the second end 606 of the housing 602. In the illustrated embodiments, the first cross-sectional shape of the first housing lumen 620 may be generally circular while the second cross-sectional shape of the first housing lumen 620 may be generally non-circular. In some cases, a portion of the second cross-sectional shape may be semi-circular a portion having linearly extending side walls similar to a portion of a stadium or capsule. However, the first cross-sectional shape and/or the second cross-sectional shape may take other cross-sectional shapes, as desired. It is further contemplated that in some embodiments, the first cross-sectional shape and the second cross-sectional shape may be same general shape. The cross-sectional dimension 616, 618 (and/or shape) of the first housing lumen 620 may change in an abrupt or step-wise manner to define a first shoulder or shoulder 626.

The housing 602 may further define an air outlet 628 extending through a side wall thereof. While the air outlet 628 is illustrated as extending through the front side wall 612, the air outlet 628 may extend through any side wall 612, 614, 646, 648 desired. In yet other embodiments, the air outlet 628 may extend through a face of the second portion 610 of the housing 606. The air outlet 628 may include a plurality of apertures 630a-e. While the first air outlet 628 is shown and described as having five apertures 630a-e, the first air outlet 628 may have fewer than five or more than five apertures, as desired. The air outlet 628 is in fluid communication with the first housing lumen 620 and is configured to be in fluid communication with the lumen of the gas supply tube 240c via the first housing lumen 620.

A one-way valve 632 (FIG. 5D) may positioned in or adjacent to the first air outlet 628. In some examples, the one-way valve 632 may be a flap valve although other one-way valves, including those described elsewhere herein, may be used, as desired. The one-way valve 632 may be configured to allow air to move from the first housing lumen 620 of the housing 602 and exit the via the apertures 630a-d as shown at arrows 634. For example, air or gas flowing through the first housing lumen 620 may deflect the flaps 636 away from the housing 602. However, the one-way valve 632 may prevent air from moving in the opposite direction. This may allow air to enter and pressurize the reservoir. The one-way valve 632 may also prevent water from entering the first housing lumen 620 of the housing 602. The one-way valve 632 may be coupled to the first air outlet 628 using a number of techniques including, but not limited to, glue, adhesives, sonic welding, ultrasound welding, etc. In some cases, a central post 638 of the one-way valve 632 may extend through the central aperture 630e to secure the one-way valve 632 to the housing 602, for example, by a snap fit or friction fit.

The gas supply tube 240c may extend into the first housing lumen 620 of housing 602, as shown in FIG. 6D. In some embodiments, at least a portion of the first end of the gas supply tube 240c may abut the first shoulder 626. However, this is not required. In some embodiments, the first end of the gas supply tube 240c may be proximal to the first shoulder 626. The gas supply tube 240c may be secured to the housing 602 using a number of techniques, including, but not limited to, friction fits, snap fits, glue, adhesive, etc. As described above, air from the air pump 215 (or gas from an alternative source) may flow through the connection portion 265 and to the reservoir 270 (depending on the position of the gas/water valve 140). As air exits the lumen of the gas supply tube 240c, the air enters the first housing lumen 620 and exits the housing 602 via the air outlet 628. The one-way valve 632 allows air to enter the reservoir 270, 305, 405 to pressurize it but does not allow for the air to re-enter the housing 602 and/or the gas supply tube 240c. It is contemplated that the first end of the gas supply tube 240c and/or the air outlet 628 may be positioned proximal to a water inlet 642 so that air may enter the housing and exit into the reservoir but not make its way up the water supply tube 245c.

The housing 602 may further include a second housing lumen 622 which may extend distally from a first end 623 distal to the first end 604 to the second end 606. In some cases, a portion of the second housing lumen 622 may begin at the second end 625 of the first housing lumen 622. In the absence of a water supply tube 245c, the second housing lumen 622 may be fluidly coupled to the first housing lumen 620. The second housing lumen 622 may vary in cross-sectional shape and/or dimension from second cross-sectional shape and/or dimension 618 of the first housing lumen 620. For example, the second housing lumen 622 may have a third cross-sectional shape having a third cross-sectional dimension 650. The third cross-sectional shape and/or third cross-sectional dimension 650 may be substantially constant along a length of the second housing lumen 622. However, this is not required. The second housing lumen 622 may vary in cross-sectional shape and/or dimension, as desired. In the illustrated embodiments, the third cross-sectional shape of the second housing lumen 622 may be generally. However, the third cross-sectional shape may take other cross-sectional shapes, as desired. The third cross-sectional dimension 650 may be less than the second cross-sectional dimension 618. However, this is not required. The cross-sectional dimension 618, 650 (and/or shape) between the first housing lumen 620 and the second housing lumen 622 may change in an abrupt or step-wise manner to define a second shoulder or ledge 652.

The second housing lumen 622 may be configured to be in fluid communication with the water supply tube 245c. In some embodiments, the water supply tube 245c may be disposed at least partially within the second housing lumen 622 to fluidly couple the lumen of the water supply tube 245c with the second housing lumen 622, as shown in FIG. 6D. In some embodiments, at least a portion of the first end of the water supply tube 245c may abut the second shoulder 650. However, this is not required. When the water supply tube 245c is fluidly coupled with the second housing lumen 622, the first housing lumen 620 and the second housing lumen 622 are fluidly isolated from one another. The water supply tube 245c may extend through a lumen of the gas supply tube 240c such that only a single opening in the cap 280, 407 is necessary. The water supply tube 245c may extend through the gas supply tube 240c such that the longitudinal axis of the water supply tube 245c is co-axial with the longitudinal axis of the gas supply tube 240c. In other examples, the water supply tube 245c and the gas supply tube 240c may extend such that the longitudinal axes thereof are laterally offset. When the reservoir 270, 305, 405 is pressurized, water may enter the housing 602 via a water inlet 642 at a distal end of the second housing lumen 622. Water may then flow through the second housing lumen 622 proximally and into the lumen of the water supply tube 245c to provide lens wash capabilities.

The housing 602 may be formed from a material that has a density greater than water. This may allow the housing 602 to act as a weight to keep the gas supply tube 240c and the water supply tube 245c from floating to the top of the reservoir 270, 305, 405. The housing 602 may begin a substantially solid member with the lumens 620, 622 and apertures 630a-e separately formed. For example, the lumens 620, 622 and apertures 630a-e may be machined into a substantially solid housing. In other examples, the housing 602 may be molded as a single monolithic structure to include the lumens 620, 622 and apertures 630a-e.

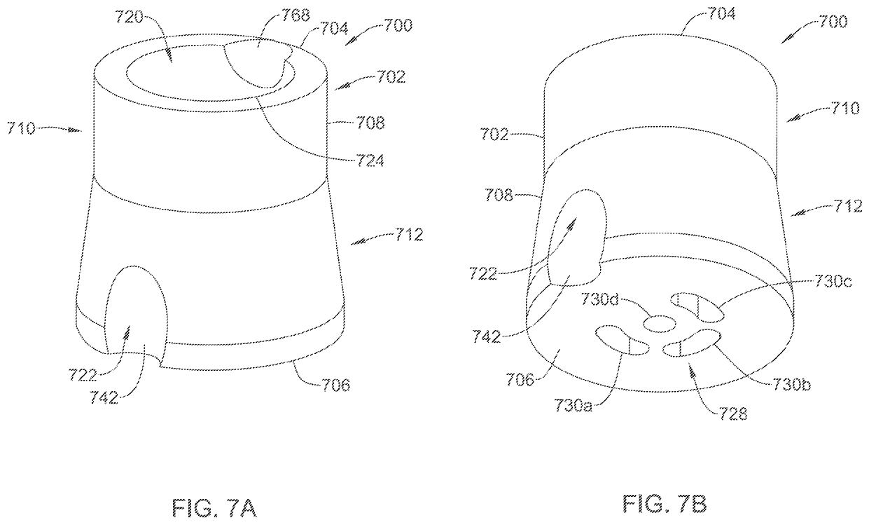

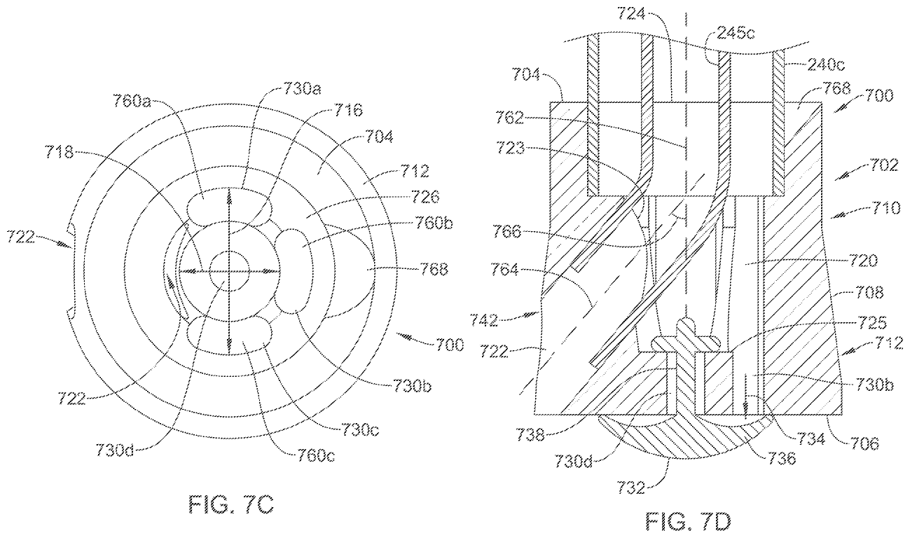

FIG. 7A depicts a top perspective view of another illustrative distal tubing weight 700 for use with the gas supply tubing 240c, the lens wash tubing 245c, and the reservoirs 270, 305, 405. FIG. 7B depicts a bottom perspective of the illustrative distal tubing weight 700. FIG. 7C depicts a top view of the illustrative distal tubing weight 700 of FIG. 7A. FIG. 7D depicts a cross-sectional view of the illustrative distal tubing weight 700 assembled with a gas supply tube 240c and a water supply tube 245c. The distal tubing weight 700 may be configured to house the gas supply tube 240c and the water supply tube 245c within the reservoir 270, 305, 405 in a configuration that reduces the complexity of the water bottle cap or top 280, 407 while also maintaining the gas supply tube 240c and water supply tube 245c in a desired configuration.

The distal tubing weight 700 includes a housing 702 extending from a first, or proximal, end 704 to a second, or distal, end 706. In some cases, the first end 704 may be considered a top of the housing 702 while the second end 706 may be considered a bottom of the housing 702. The illustrative housing 702 may have a generally cylindrical structure. However, the housing 702 is not limited to a cylindrical structure. Other shapes or combinations of shapes may be used for the housing 702, as desired, including but not limited to a cubic structure, rectangular or generally rectangular structure, or a pyramidal structure. The housing 702 includes a circumferentially extending side wall 708. In some embodiments, the housing 702 may have a first portion 70 having a substantially constant outer diameter and a second portion 712 having an outer diameter that increases in the distal direction. However, this is not required. In some cases, the outer diameter of the housing 702 may be substantially constant from the first end 704 to the second end 706. In yet other embodiments, the outer diameter may increase or taper from the first end 704 to the second end 706.

The housing 702 may define a first housing lumen 720 extending distally from the first end 704 to a point proximal to the second end 706. The first housing lumen 720 may define an opening 724 in the first end 704 of the housing 702 and terminate at a second end 725 proximal to the second end 706 of the housing 702. The first housing lumen 720 may vary in cross-sectional shape and/or dimension along a length thereof. For example, the first housing lumen 720 may have a first cross-sectional shape having a first cross-sectional dimension 716 adjacent to the first end 704 of the housing 702 and a second cross-sectional shape having a second cross-sectional dimension 718 adjacent to the second end 706 of the housing 702. In the illustrated embodiments, the first cross-sectional shape of the first housing lumen 720 may be generally circular while the second cross-sectional shape of the first housing lumen 720 may be also be generally circular. However, the first cross-sectional shape and/or the second cross-sectional shape may take other cross-sectional shapes, as desired. It is further contemplated that in some embodiments, the first cross-sectional shape and the second cross-sectional shape may be differing shapes. The cross-sectional dimension 716, 718 (and/or shape) of the first housing lumen 720 may change in an abrupt or step-wise manner to define a first shoulder or ledge 726.

The housing 702 may further define an air outlet 728 extending through the bottom 706 of the housing 702. However, the air outlet 728 may extend through the side wall 708 if so desired. The air outlet 728 may include a plurality of apertures 730a-d. While the first air outlet 728 is shown and described as having four apertures 730a-d, the first air outlet 728 may have fewer than four or more than four apertures, as desired. The air outlet 728 is in fluid communication with the first housing lumen 720 and is configured to be in fluid communication with the lumen of the gas supply tube 240c via the first housing lumen 720. In some embodiments, at least some of the apertures 730a-c may extend proximally from the second end 706 to the shoulder 726 to create air flow channels 760a-c. It is contemplated that the air flow channels 730a-c may form a part of the second cross-sectional shape of the first housing lumen 720. In such an instance, the second cross-sectional shape may be non-circular.

A one-way valve 732 (FIG. 7D) may positioned in or adjacent to the first air outlet 728. In some examples, the one-way valve 732 may be a flap valve although other one-way valves, including those described elsewhere herein, may be used, as desired. The one-way valve 732 may be configured to allow air to move from the first housing lumen 720 of the housing 702 and exit the via the apertures 730a-d as shown at arrow 734. For example, air or gas flowing through the first housing lumen 720 may deflect the flaps 736 away from the housing 702. However, the one-way valve 732 may prevent air from moving in the opposite direction. This may allow air to enter and pressurize the reservoir. The one-way valve 732 may also prevent water from entering the first housing lumen 720 of the housing 702. The one-way valve 732 may be coupled to the first air outlet 728 using a number of techniques including, but not limited to, glue, adhesives, sonic welding, ultrasound welding, etc. In some cases, a central post 738 of the one-way valve 732 may extend through the central aperture 730d to secure the one-way valve 732 to the housing 702, for example, by a snap fit or friction fit.

The gas supply tube 240c may extend into the first housing lumen 720 of housing 702, as shown in FIG. 7D. In some embodiments, at least a portion of the first end of the gas supply tube 240c may abut the first shoulder 726. However, this is not required. In some embodiments, the first end of the gas supply tube 240c may be proximal to the first shoulder 726. The gas supply tube 240c may be secured to the housing 702 using a number of techniques, including, but not limited to, friction fits, snap fits, glue, adhesive, etc. As described above, air from the air pump 215 (or gas from an alternative source) may flow through the connection portion 265 and to the reservoir 270 (depending on the position of the gas/water valve 140). As air exits the lumen of the gas supply tube 240c, the air enters the first housing lumen 720 and exits the housing 702 via the air outlet 728. The one-way valve 732 allows air to enter the reservoir 270, 305, 405 to pressurize it but does not allow for the air to re-enter the housing 702 and/or the gas supply tube 240c. It is contemplated that the first end of the gas supply tube 240c and/or the air outlet 728 may be positioned proximal to a water inlet 742 so that air may enter the housing and exit into the reservoir but not make its way up the water supply tube 245c.

The housing 702 may further include a notch or recess 768 formed in the first end 704 of the housing 702 at the opening 724. The recess 768 may be curved to provide a lead-in feature for the gas supply tube 240c and/or water supply tube 245c. This may help prevent kinking of the gas supply tube 240c and/or water supply tube 245c.

The housing 702 may further include a second housing lumen 722 which may extend distally from a first end 723 distal to the first end 704 towards a fluid outlet 742 extending at least partially through the side wall 708 of the housing 702. The second housing lumen 722 may extend along a longitudinal axis 764 that extends at an angle 766 to a longitudinal axis 762 of the first housing lumen 720. The angle 766 may be generally non-orthogonal and may be in the range of greater than 0° to about less than 90°. In the absence of a water supply tube 245c, the second housing lumen 722 may be fluidly coupled to the first housing lumen 720.

The second housing lumen 722 may be configured to be in fluid communication with the water supply tube 245c. In some embodiments, the water supply tube 245c may be disposed at least partially within the second housing lumen 722 to fluidly couple the lumen of the water supply tube 245c with the second housing lumen 722, as shown in FIG. 7D. When the water supply tube 245c is fluidly coupled with the second housing lumen 722, the first housing lumen 720 and the second housing lumen 722 are fluidly isolated from one another. The water supply tube 245c may extend through a lumen of the gas supply tube 240c such that only a single opening in the cap 280, 407 is necessary. When the reservoir 270, 305, 405 is pressurized, water may enter the housing 702 via a water inlet 742 at a distal end of the second housing lumen 722. Water may then flow through the second housing lumen 722 proximally and into the lumen of the water supply tube 245c to provide lens wash capabilities.

The housing 702 may be formed from a material that has a density greater than water. This may allow the housing 702 to act as a weight to keep the gas supply tube 240c and the water supply tube 245c from floating to the top of the reservoir 270, 305, 405. The housing 702 may begin a substantially solid member with the lumens 720, 722 and apertures 730a-d separately formed. For example, the lumens 720, 722 and apertures 730a-d may be machined into a substantially solid housing. In other examples, the housing 702 may be molded as a single monolithic structure to include the lumens 720, 722 and apertures 730a-d.

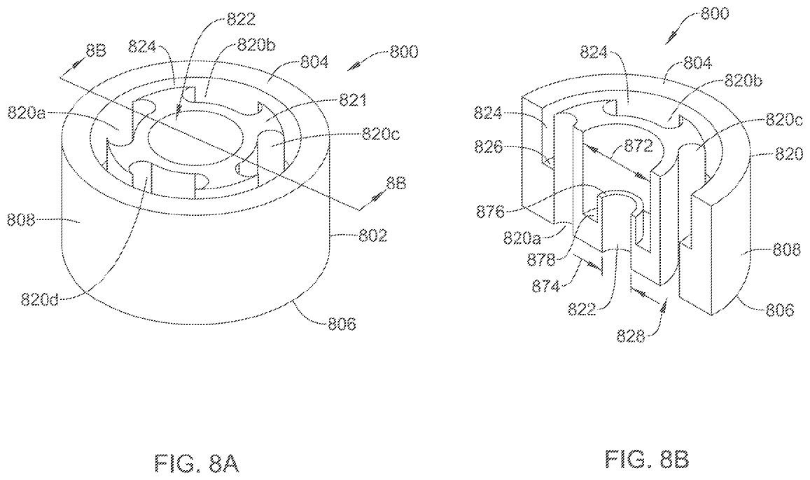

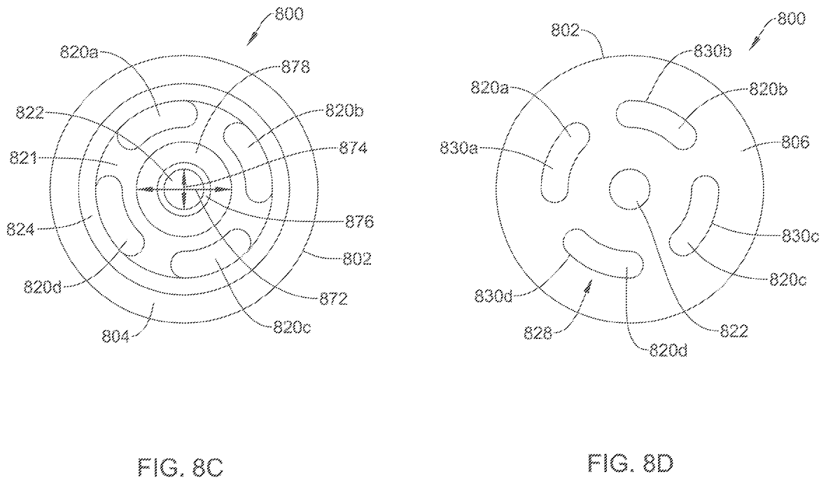

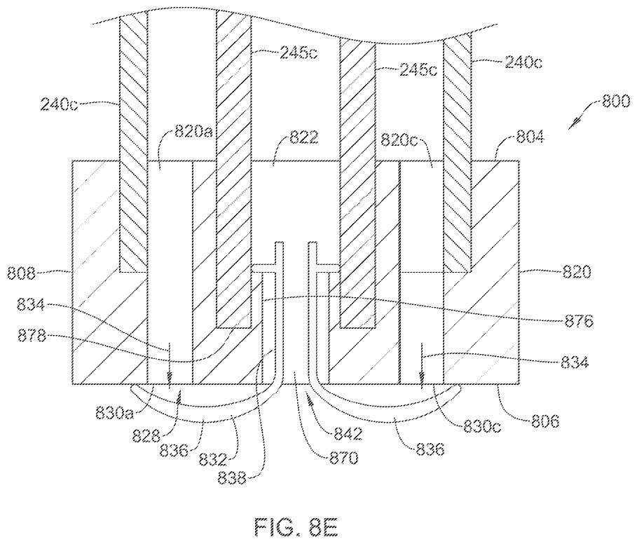

FIG. 8A depicts a top perspective view of another illustrative distal tubing weight 800 for use with the gas supply tubing 240c, the lens wash tubing 245c, and the reservoirs 270, 305, 405. FIG. 8B depicts a perspective cross-sectional view of the illustrative distal tubing weight 800 taken at line 8B-8B of FIG. 8A. FIG. 8C depicts a top view of the illustrative distal tubing weight 800 of FIG. 8A. FIG. 8D depicts a bottom view of the illustrative distal tubing weight 800 of FIG. 8A. FIG. 8E depicts a cross-sectional view of the illustrative distal tubing weight 800 assembled with a gas supply tube 240c and a water supply tube 245c. The distal tubing weight 800 may be configured to house the gas supply tube 240c and the water supply tube 245c within the reservoir 270, 305, 405 in a configuration that reduces the complexity of the water bottle cap or top 280, 407 while also maintaining the gas supply tube 240c and water supply tube 245c in a desired configuration.

The distal tubing weight 800 includes a housing 802 extending from a first, or proximal, end 804 to a second, or distal, end 806. In some cases, the first end 804 may be considered a top of the housing 802 while the second end 806 may be considered a bottom of the housing 802. The illustrative housing 802 may have a generally cylindrical structure. However, the housing 802 is not limited to a cylindrical structure. Other shapes or combinations of shapes may be used for the housing 802, as desired, including but not limited to a cubic structure, rectangular or generally rectangular structure, or a pyramidal structure. The housing 802 includes a circumferentially extending side wall 808. In some cases, the outer diameter of the housing 802 may be substantially constant from the first end 804 to the second end 806. In yet other embodiments, the outer diameter may increase or taper from the first end 804 to the second end 806.

The housing 802 may define a first housing lumen formed from a plurality of channels 820a-d extending distally from the first end 804 to the second end 806. An annular lumen 824 may be positioned radially outward from the channels 820a-d. The annular lumen 824 may extend distally from the first end 804 to a point proximal to the second end 806. The annular lumen 824 may terminate at a shoulder or shelf 826. The annular lumen 824 may be configured to receive the first end of the gas supply tube 240c. However, in the absence of the gas supply tube 240c, the annular lumen 824 may be fluidly coupled to the plurality of channels 820a-d. The plurality of channels 820a-d may each have a uniform cross-sectional shape from the first end 804 of the housing 802 to the second end 806 of the housing 802. In other embodiments, the cross-sectional shape and/or dimension of one or more of the plurality of channels 820a-d may vary along a length thereof.

The housing 802 may further define an air outlet 828 extending through the bottom 806 of the housing 802. The air outlet 828 may be formed from the second ends 830a-d of the plurality of channels 820a-d. While the first air outlet 828 is shown and described as having four channels 820a-d, the first air outlet 828 may have fewer than four or more than four channels 820a-d, as desired. The air outlet 828 is in fluid communication with the plurality of channels 820a-d and is configured to be in fluid communication with the lumen of the gas supply tube 240c via the plurality of channels 820a-d.

A one-way valve 832 (FIG. 8E) may positioned in or adjacent to the first air outlet 828. In some examples, the one-way valve 832 may be a flap valve although other one-way valves, including those described elsewhere herein, may be used, as desired. The one-way valve 832 may be configured to allow air to move from the plurality of channels 820a-d of the housing 802 and exit the via the second ends 830a-d as shown at arrows 834. For example, air or gas flowing through the plurality of channels 820a-d may deflect the flaps 836 away from the housing 802. However, the one-way valve 832 may prevent air from moving in the opposite direction. This may allow air to enter and pressurize the reservoir. The one-way valve 832 may also prevent water from entering the plurality of channels 820a-d of the housing 802. The one-way valve 832 may be coupled to the first air outlet 828 using a number of techniques including, but not limited to, glue, adhesives, sonic welding, ultrasound welding, etc. In some cases, a central post 838 of the one-way valve 832 may extend through a second housing lumen 822 to secure the one-way valve 832 to the housing 802, for example, by a snap fit or friction fit. As will be described in more detail herein, the central post 838 may define a lumen 870 to allow fluid to enter the second housing lumen 822 and the water supply tube 245c.

The gas supply tube 240c may extend into the annular lumen 824 of the housing 802, as shown in FIG. 8E. In some embodiments, at least a portion of the first end of the gas supply tube 240c may abut the first shoulder 826. However, this is not required. In some embodiments, the first end of the gas supply tube 240c may be proximal to the first shoulder 826. An inner surface of the gas supply tube 240c may contact a body portion of the housing 802 generally disposed between the plurality of channels 820a-d. The body portion 821 may be generally solid and be configured to fluidly isolate the plurality of channels 820a-d from the second housing lumen 822. The gas supply tube 240c may be secured to the housing 802 using a number of techniques, including, but not limited to, friction fits, snap fits, glue, adhesive, etc. As described above, air from the air pump 215 (or gas from an alternative source) may flow through the connection portion 265 and to the reservoir 270 (depending on the position of the gas/water valve 140). As air exits the lumen of the gas supply tube 240c, the air enters the plurality of channels 820a-d and exits the housing 802 via the air outlet 728. The one-way valve 832 allows air to enter the reservoir 270, 305, 405 to pressurize it but does not allow for the air to re-enter the housing 802 and/or the gas supply tube 240c. It is contemplated that the first end of the gas supply tube 240c and/or the air outlet 828 may be positioned relative to a water inlet 842 so that air may enter the housing 802 and exit into the reservoir but not make its way up the water supply tube 245c.

The housing 802 may further include a second housing lumen 822 which may extend distally from the first end 804 of the housing 802 towards a fluid outlet 842 at the second end 806 of the housing 802. The second housing lumen 822 may vary in cross-sectional shape and/or dimension along a length thereof. For example, the second housing lumen 822 may have a first cross-sectional shape having a first cross-sectional dimension 872 adjacent to the first end 804 of the housing 802 and a second cross-sectional shape having a second cross-sectional dimension 874 adjacent to the second end 806 of the housing 802. In the illustrated embodiments, the first and second cross-sectional shapes of the second housing lumen 822 may be generally circular. However, the first cross-sectional shape and/or the second cross-sectional shape may take other cross-sectional shapes, as desired. The second cross-sectional dimension 874 may be less than the first cross-sectional dimension 874. The cross-sectional dimension 872, 874 (and/or shape) of the second housing lumen 822 may change in an abrupt or step-wise manner to define a second shoulder or ledge 878. In some cases, a portion of the second housing lumen 822 may be defined by a tubular member 876 extending proximally from the second shoulder 878. However, this is not required. The tubular member 876 may increase in outer diameter in the distal direction. While this is not required, the increasing diameter may facilitate coupling of the water supply tube 245c with the tubular member 876.

The second housing lumen 822 may be configured to be in fluid communication with the water supply tube 245c. In some embodiments, the water supply tube 245c may be disposed over the tubular member 876 to fluidly couple the lumen of the water supply tube 245c with the second housing lumen 822, as shown in FIG. 8E. In some embodiments, at least a portion of the first end of the water supply tube 245c may abut the second shoulder 878. However, this is not required. In other embodiments, the first end of the water supply tube 245c may be inserted into the tubular member 876. When the water supply tube 245c is fluidly coupled with the second housing lumen 822, the plurality of channels 820a-d and the second housing lumen 822 are fluidly isolated from one another. The water supply tube 245c may extend through a lumen of the gas supply tube 240c such that only a single opening in the cap 280, 407 is necessary. The water supply tube 245c may extend through the gas supply tube 240c such that the longitudinal axis of the water supply tube 245c is co-axial with the longitudinal axis of the gas supply tube 240c. In other examples, the water supply tube 245c and the gas supply tube 240c may extend such that the longitudinal axes thereof are laterally offset. When the reservoir 270, 305, 405 is pressurized, water may enter the housing 802 via a water inlet 842 at a distal end of the second housing lumen 822. Water may then flow through lumen 870 of the valve 832 and into the lumen of the water supply tube 245c to provide lens wash capabilities.

The housing 802 may be formed from a material that has a density greater than water. This may allow the housing 802 to act as a weight to keep the gas supply tube 240c and the water supply tube 245c from floating to the top of the reservoir 270, 305, 405. The housing 802 may begin a substantially solid member with the plurality of channels 820a-d and lumens 822, 824 separately formed. For example, the plurality of channels 820a-d and lumens 822, 824 may be machined into a substantially solid housing. In other examples, the housing 802 may be molded as a single monolithic structure to include the plurality of channels 820a-d and lumens 822, 824.

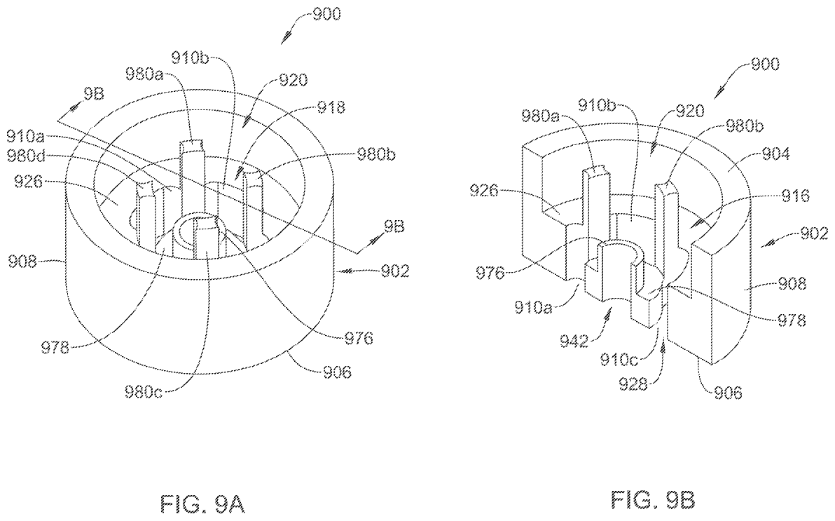

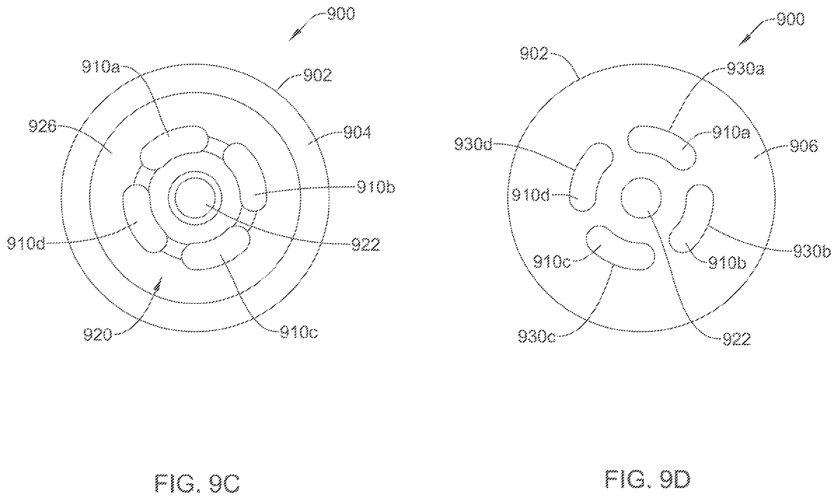

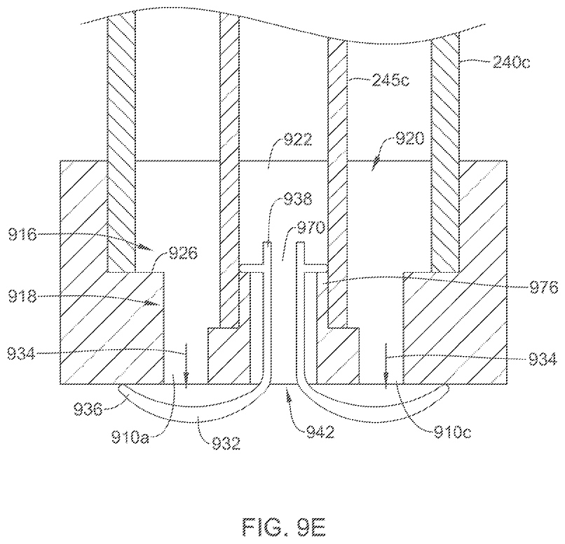

FIG. 9A depicts a top perspective view of another illustrative distal tubing weight 900 for use with the gas supply tubing 240c, the lens wash tubing 245c, and the reservoirs 270, 305, 405. FIG. 9B depicts a perspective cross-sectional view of the illustrative distal tubing weight 900 taken at line 9B-9B of FIG. 9A. FIG. 9C depicts a top view of the illustrative distal tubing weight 900 of FIG. 9A. FIG. 9D depicts a bottom view of the illustrative distal tubing weight 900 of FIG. 9A. FIG. 9E depicts a cross-sectional view of the illustrative distal tubing weight 900 assembled with a gas supply tube 240c and a water supply tube 245c. The distal tubing weight 900 may be configured to house the gas supply tube 240c and the water supply tube 245c within the reservoir 270, 305, 405 in a configuration that reduces the complexity of the water bottle cap or top 290, 407 while also maintaining the gas supply tube 240c and water supply tube 245c in a desired configuration.

The distal tubing weight 900 includes a housing 902 extending from a first, or proximal, end 904 to a second, or distal, end 906. In some cases, the first end 904 may be considered a top of the housing 902 while the second end 906 may be considered a bottom of the housing 902. The illustrative housing 902 may have a generally cylindrical structure. However, the housing 902 is not limited to a cylindrical structure. Other shapes or combinations of shapes may be used for the housing 902, as desired, including but not limited to a cubic structure, rectangular or generally rectangular structure, or a pyramidal structure. The housing 902 includes a circumferentially extending side wall 908. In some cases, the outer diameter of the housing 902 may be substantially constant from the first end 904 to the second end 906. In yet other embodiments, the outer diameter may increase or taper from the first end 904 to the second end 906.

The housing 902 may define a first housing lumen 920 including a first portion 916 and a second portion 918 including a plurality of channels 910a-d. The first housing lumen 920 may extend distally from the first end 904 to the second end 906 of the housing 902. The first housing lumen 920 may vary in cross-sectional shape and/or dimension along a length thereof. For example, the first portion 916 of the first housing lumen 920 may have a first cross-sectional shape having a first cross-sectional dimension adjacent to the first end 904 of the housing 902 and the second portion 918 may have a second cross-sectional shape having a second cross-sectional dimension adjacent to the second end 906 of the housing 902. In the illustrated embodiments, the first cross-sectional shape of the first housing lumen 920 may be generally circular while the second cross-sectional shape of the first housing lumen 920 may have a plurality of curved oblong shapes. However, the first cross-sectional shape and/or the second cross-sectional shape may take other cross-sectional shapes, as desired. It is further contemplated that in some embodiments, the first cross-sectional shape and the second cross-sectional shape may be same general shape. The second cross-sectional dimension may be less than the first cross-sectional dimension. The first portion 916 of the first housing lumen 920 may transition to the second portion 918 of the first housing lumen 920 in abrupt manner to define a first ledge or shoulder 926. The first portion 916 of the first housing lumen 920 may be configured to receive the gas supply tube 240c. The plurality of channels 910a-d may each have a uniform cross-sectional shape from the first shoulder 926 of the housing 902 to the second end 906 of the housing 902. In other embodiments, the cross-sectional shape and/or dimension of one or more of the plurality of channels 910a-d may vary along a length thereof.

The housing 902 may further define an air outlet 928 extending through the bottom 906 of the housing 902. The air outlet 928 may be formed from the second ends 930a-d of the plurality of channels 910a-d. While the first air outlet 928 is shown and described as having four channels 910a-d, the first air outlet 928 may have fewer than four or more than four channels 910a-d, as desired. The air outlet 928 is in fluid communication with the plurality of channels 910a-d and is configured to be in fluid communication with the lumen of the gas supply tube 240c via the plurality of channels 910a-d and/or the first housing lumen 920.

A one-way valve 932 (FIG. 9E) may positioned in or adjacent to the first air outlet 928. In some examples, the one-way valve 932 may be a flap valve although other one-way valves, including those described elsewhere herein, may be used, as desired. The one-way valve 932 may be configured to allow air to move from the plurality of channels 910a-d of the housing 902 and exit the via the second ends 930a-d as shown at arrows 934. For example, air or gas flowing through the plurality of channels 910a-d may deflect the flaps 936 away from the housing 902. However, the one-way valve 932 may prevent air from moving in the opposite direction. This may allow air to enter and pressurize the reservoir. The one-way valve 932 may also prevent water from entering the plurality of channels 910a-d of the housing 902. The one-way valve 932 may be coupled to the first air outlet 928 using a number of techniques including, but not limited to, glue, adhesives, sonic welding, ultrasound welding, etc. In some cases, a central post 938 of the one-way valve 932 may extend through a second housing lumen 922 to secure the one-way valve 932 to the housing 902, for example, by a snap fit or friction fit. As will be described in more detail herein, the central post 938 may define a lumen 970 to allow fluid to enter the second housing lumen 922 and the water supply tube 245c.

The gas supply tube 240c may extend into the first portion 916 of the first housing lumen 920 of the housing 902, as shown in FIG. 9E. In some embodiments, at least a portion of the first end of the gas supply tube 240c may abut the first shoulder 926. As the housing 902 does not include a physical structure to fluidly isolate the gas supply tube 240c and the water supply tube 245c, the distal face of the gas supply tube 240c may be secured to the shoulder 926 to provide an air-tight seal to ensure air does not leak into the water supply tube 245c. The gas supply tube 240c may be secured to the housing 902 using a number of techniques, including, but not limited to, friction fits, snap fits, glue, adhesive, etc. As described above, air from the air pump 215 (or gas from an alternative source) may flow through the connection portion 265 and to the reservoir 270 (depending on the position of the gas/water valve 140). As air exits the lumen of the gas supply tube 240c, the air enters the plurality of channels 910a-d and exits the housing 902 via the air outlet 728. The one-way valve 932 allows air to enter the reservoir 270, 305, 405 to pressurize it but does not allow for the air to re-enter the housing 902 and/or the gas supply tube 240c. It is contemplated that the first end of the gas supply tube 240c and/or the air outlet 928 may be positioned relative to a water inlet 942 so that air may enter the housing 902 and exit into the reservoir but not make its way up the water supply tube 245c.

The housing 902 may further include a second housing lumen 922 which may extend distally from a point distal to the first end 904 to the water inlet 942 at the second end 906. In some cases, a portion of the second housing lumen 922 may be defined by a tubular member 976 extending proximally from a second shoulder 978. However, this is not required. The tubular member 976 may increase in outer diameter in the distal direction. While this is not required, the increasing diameter may facilitate coupling of the water supply tube 245c with the tubular member 976.

The second housing lumen 922 may be configured to be in fluid communication with the water supply tube 245c. In some embodiments, the water supply tube 245c may be disposed over the tubular member 976 to fluidly couple the lumen of the water supply tube 245c with the second housing lumen 922, as shown in FIG. 9E. The distal face of the water supply tube 245c may be secured to the second shoulder 978 to provide a fluid and air-tight seal to ensure water does not leak into the gas supply tube 240c. In other embodiments, the first end of the water supply tube 245c may be inserted into the tubular member 976. When the water supply tube 245c is fluidly coupled with the second housing lumen 922, the first housing lumen 920 and the second housing lumen 922 are fluidly isolated from one another. The water supply tube 245c may extend through a lumen of the gas supply tube 240c such that only a single opening in the cap 290, 407 is necessary. The water supply tube 245c may extend through the gas supply tube 240c such that the longitudinal axis of the water supply tube 245c is co-axial with the longitudinal axis of the gas supply tube 240c. In other examples, the water supply tube 245c and the gas supply tube 240c may extend such that the longitudinal axes thereof are laterally offset. When the reservoir 270, 305, 405 is pressurized, water may enter the housing 902 via a water inlet 942 at a distal end of the second housing lumen 922. Water may then flow through lumen 970 of the valve 932 and into the lumen of the water supply tube 245c to provide lens wash capabilities.

The housing 902 may further include a plurality of posts 980a-d positioned radially spaced from the tubular member 976. The posts 980a-d may extend proximally from the second end 906 of the housing 906. The posts may be configured to support the water supply tube 245c to maintain a position of the water supply tube 245c. For example, the posts 980a-d may have a surface configured to contact and conform to an outer surface of the water supply tube 245c. While the housing 902 is illustrated as including four posts 980a-d, the housing 902 may include fewer than four or more than four posts, as desired.

The housing 902 may be formed from a material that has a density greater than water. This may allow the housing 902 to act as a weight to keep the gas supply tube 240c and the water supply tube 245c from floating to the top of the reservoir 270, 305, 405. The housing 902 may begin a substantially solid member with the plurality of channels 910a-d and lumens 920, 922 separately formed. For example, the plurality of channels 910a-d and lumens 920, 922 may be machined into a substantially solid housing. In other examples, the housing 902 may be molded as a single monolithic structure to include the plurality of channels 910a-d and lumens 920, 922.

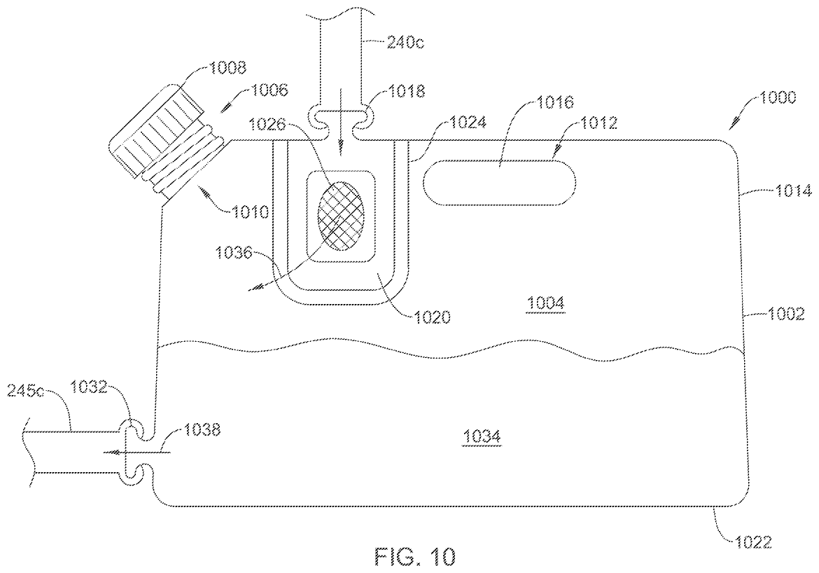

FIG. 10 depicts a side view of an illustrative refillable fluid reservoir 1000. The reservoir 1000 may be configured to be used in an endoscopic system and includes components similar to the endoscope and endoscope systems described with regard to FIGS. 1-4; however, not all features may be described or shown here if not pertinent to the fluid circuit of the system. The reservoir 1000 may be configured to couple to the gas supply tube 240c and the water supply tube 245c in a configuration that reduces the complexity of the water bottle cap or top 280, 407 while also maintaining the gas supply tube 240c and water supply tube 245c in a desired configuration.

The reservoir 1000 includes a container 1002 defining a first receptacle 1004 configured to hold a fluid 1034. The container 1002 may be formed from a lightweight, flexible material, such as, but not limited to low density polyethylene (LDPE), thermoplastic polyurethane (TPU), silicone, polyethylene terephthalate (PET), aluminum, nylon, polyethylene (PE), or combinations thereof, etc. In some embodiments, the container 1002 may be entirely translucent, entirely opaque, or combinations thereof. The reservoir 1000 may further include a port 1006 having a removable cap 1008. The cap 1008 may be formed from more rigid material (relative to the container 1002) and may be configured to form a fluid tight seal with the port 1006. The cap 1008 may be configured to threadably engage the port 1006, form a friction fit with the port 1006, form a snap fit with the port 1006 or otherwise releasably engage the port 1006. In some examples, the port 1006 and/or cap 1008 may be formed from polyethylene terephthalate (PET), polypropylene (PP), etc. Portions of the port 1006 may extend into the first receptacle 1004. The removable cap 1008 may be removed to place a fluid source in selective fluid communication with the first receptacle 1004 and allow fluid to be poured through a lumen 1010 of the port 1006 and into the first receptacle 1004.

The reservoir 1000 may include a carrying handle 1012 positioned adjacent to a top portion 1014 thereof. The handle 1012 may define an opening or through hole 1016 for receiving a hand or hook therethrough to carry the reservoir 1000. In some cases, the carrying handle 1012 may include an undulating carrying surface (not explicitly shown) configured to provide a more ergonomic grip for the user. It is contemplated that the handle 1012 may be formed from a similar material as the cap 1008 or the container 1002, as desired. In some examples, the handle 1012 may be formed from polyethylene terephthalate (PET), polypropylene (PP), etc.

The reservoir 1000 may be movable between a collapsed storage configuration (not explicitly shown) and an expanded use configuration (FIG. 10). In the expanded use configuration, the reservoir 1000 may increase in width from the top portion 1014 towards the bottom portion 1022. In the use configuration, the bottom portion 1022 may have a width that allows the reservoir 1000 to remain upright without user intervention. The bottom portion 1022 may include folds or pleats that allow the bottom portion 1022 to fold or collapse. In the collapsed storage configuration, the top portion 1014 and the bottom portion 1022 may have a similar width which allows the reservoir 1000 to lay substantially flat such that the reservoir 1000 may be stacked with other fluid reservoirs 1000. In other examples, the reservoir 1000 may be rolled or folded to reduce the amount of storage space it occupies. In some cases, since the reservoir 1000 is sealed or capable of being sealed, a vacuum may be pulled during packaging of the reservoir 1000 to further reduce the storage space required to store the reservoir 1000.

The reservoir 1000 may be connected in fluid communication with a gas supply/alternate gas supply tubing (or gas supply tubing) 240c and lens wash supply/irrigation supply tubing 245c (or water supply tubing 245c). The gas supply tubing 240c extends from a second end external to the reservoir 1000 to a first end coupled to a coupling mechanism or adaptor 1018. A lumen extends through the gas supply tubing 240c for receiving a flow of air and/or gas therethrough. The lumen of the gas supply tubing 240c is in operative fluid communication with the interior of the reservoir 1000. The adaptor 1018 may be positioned adjacent to the top portion 1014 of the container 1002. However, this is not required. The adaptor 1018 may be positioned at any location desired. The adaptor 1018 is configured to fluidly couple the gas supply tubing with an inner chamber 1020 positioned within the first receptacle 1004. The inner chamber 1020 may be formed from a similar material as the container 1002 and may form a separate chamber from the first receptacle 1004. In some embodiments, the edges 1024 of the inner chamber 1020 may be heat-sealed to the first receptacle 1004 to maintain the orientation of the inner chamber 1020 relative to the first receptacle 1004. The inner chamber 1020 may further include a hydrophobic membrane 1026. In use, the hydrophobic membrane 1026 may allow air/gas to pass from the inner chamber 1020 to the first receptacle 1004, as shown at arrow 1036, to pressurize the first receptacle 1004 while preventing water from flowing into the inner chamber 1020.

The water supply tubing 245c extends from a second end external to the reservoir 1000 to a first end coupled to a second coupling mechanism or adaptor 1032. The second adaptor 1032 may be positioned adjacent to the bottom portion 1022 of the container 1002 so that fluid readily flows from the first receptacle to the water supply tubing 245c when the container 1002 is pressurized. A lumen extends through the water supply tubing 245c for receiving a flow of fluid therethrough, as shown at arrow 1038. The lumen of the lens wash supply/irrigation supply tubing 245c is in selective operative fluid communication with the bottom portion of the container 1002. In the illustrated embodiment, the gas supply tubing 240c and the water supply tubing 245c may enter the container 1002 through separate adaptors 1018, 1032. However, in some embodiments, the water supply tubing 245c may enter through other parts of the container 1002 such as, but not limited to a top portion 1014 thereof. In such an instance, the water supply tubing 245c may include a dip tube which extends to the bottom portion 1022 of the container 1002.

A portion of the gas supply tubing 240c and a portion of the water supply tubing 245c may extend from the container 1002, and may be connected in fluid communication with the endoscope at gas/lens wash connection on the connector portion 265 of the umbilical. The portion of the gas supply tubing 240c is connected in fluid communication with a gas pump (not explicitly shown) and gas feed line (not explicitly shown), and the portion of the lens wash supply tubing 245c is connected in fluid communication with lens wash feed line (not explicitly shown), within connector portion 265. While not explicitly shown, irrigation supply tubing may be coupled to the container 1002 via a separate adaptor or port to supply irrigation fluid from the reservoir 1000.

It is contemplated that the reservoir 1000 may be filled and refilled as needed by removing the cap 1008 and pouring water into the first receptacle 1004. The refilling of the reservoir 1000 may be performed during a procedure or between procedures, as necessary. The water may be sterile or non-sterile, as desired. For example, sterile water may be used for therapeutic procedures while non-sterile water may be used for diagnostic procedures. It is contemplated that refilling the reservoir 1000 with sterile or non-sterile water may create more flexibility and reduce the need to have as much sterile water in storage. Further, refilling the reservoir 1000 via the port 1006 and removable cap 1008 may also remove the need to disconnect the reservoir 1000 from the tubing 240c, 245c throughout the day eliminating or greatly reducing the possibility of cross contamination by removing the need to replace the water container.

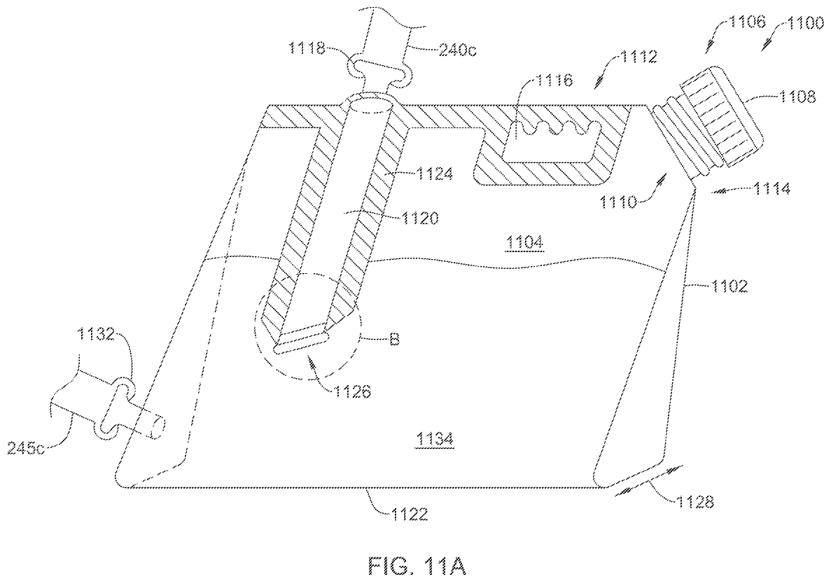

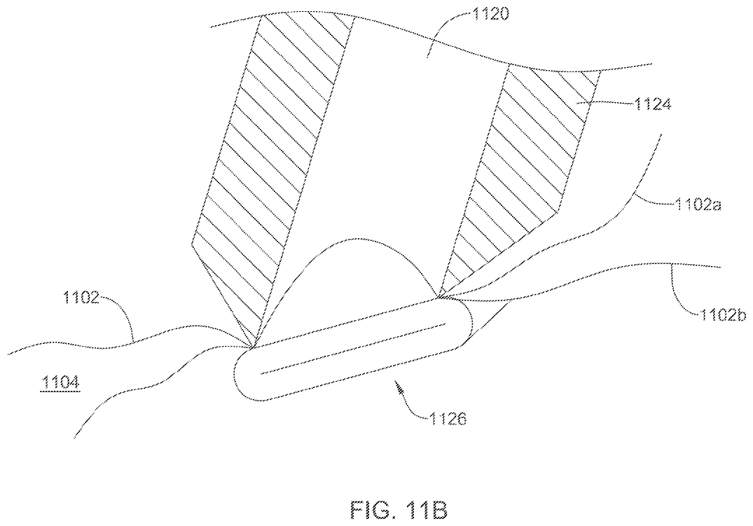

FIG. 11A depicts a side view of another illustrative refillable fluid reservoir 1100 and tube set. The reservoir 1100 may be configured to be used in an endoscopic system and includes components similar to the endoscope and endoscope systems described with regard to FIGS. 1-4; however, not all features may be described or shown here if not pertinent to the fluid circuit of the system. The reservoir 1100 may be configured to couple to the gas supply tube 240c and the water supply tube 245c in a configuration that reduces the complexity of the water bottle cap or top 280, 407 while also maintaining the gas supply tube 240c and water supply tube 245c in a desired configuration.

The reservoir 1100 includes a container 1102 defining a first receptacle 1104 configured to hold a fluid 1134. The container 1102 may be formed from a lightweight, flexible material, such as, but not limited to low density polyethylene (LDPE), thermoplastic polyurethane (TPU), silicone, polyethylene terephthalate (PET), aluminum, nylon, polyethylene (PE), or combinations thereof, etc. In some embodiments, the container 1102 may be entirely translucent, entirely opaque, or combinations thereof. The reservoir 1100 may further include a port 1106 having a removable cap 1108. The cap 1108 may be formed from more rigid material (relative to the container 1102) and may be configured to form a fluid tight seal with the port 1106. The cap 1108 may be configured to threadably engage the port 1106, form a friction fit with the port 1106, form a snap fit with the port 1106 or otherwise releasably engage the port 1106. In some examples, the port 1106 and/or cap 1108 may be formed from polyethylene terephthalate (PET), polypropylene (PP), etc. Portions of the port 1106 may extend into the first receptacle 1104. The removable cap 1108 may be removed to place a fluid source in selective fluid communication with the first receptacle 1104 and allow fluid to be poured through a lumen 1110 of the port 1106 and into the first receptacle 1104.

The reservoir 1100 may include a carrying handle 1112 positioned adjacent to a top portion 1114 thereof. The handle 1112 may define an opening or through hole 1116 for receiving a hand or hook therethrough to carry the reservoir 1100. In some cases, the carrying handle 1112 may include an undulating carrying surface configured to provide a more ergonomic grip for the user. In some embodiments, the handle 1112 may be formed from the container 1102. For example, opposing sides of the container 1112 may be heat-sealed at the desired location of the handle 1112. The opening 1116 may then be formed by removing some of the heat-sealed area. In other embodiments the handle 1112 may be separately formed from a similar material as the cap 1108 or the container 1102, as desired and coupled to the container 1112. In some examples, the handle 1112 may be formed from polyethylene terephthalate (PET), polypropylene (PP), etc.

The reservoir 1100 may be movable between a collapsed storage configuration (not explicitly shown) and an expanded use configuration (FIG. 11A). In the expanded use configuration, the reservoir 1100 may increase in width from the top portion 1114 towards the bottom portion 1122. In the use configuration, the bottom portion 1122 may have a width 1128 that allows the reservoir 1100 to remain upright without user intervention. The bottom portion 1122 may include folds or pleats that allow the bottom portion 1122 to fold or collapse. In the collapsed storage configuration, the top portion 1114 and the bottom portion 1122 may have a similar width which allows the reservoir 1100 to lay substantially flat such that the reservoir 1100 may be stacked with other fluid reservoirs 1100. In other examples, the reservoir 1100 may be rolled or folded to reduce the amount of storage space it occupies. In some cases, since the reservoir 1100 is sealed or capable of being sealed, a vacuum may be pulled during packaging of the reservoir 1100 to further reduce the storage space required to store the reservoir 1100.

The reservoir 1100 may be connected in fluid communication with a gas supply/alternate gas supply tubing (or gas supply tubing) 240c and lens wash supply/irrigation supply tubing 245c (or water supply tubing 245c). The gas supply tubing extends from a second end external to the reservoir 1100 to a first end coupled to a coupling mechanism or adaptor 1118. A lumen extends through the gas supply tubing 240c for receiving a flow of air and/or gas therethrough. The lumen of the gas supply tubing 240c is in operative fluid communication with the interior of the reservoir 1100. The adaptor 1118 may be positioned adjacent to the top portion 1114 of the container 1102. However, this is not required. The adaptor 1118 may be positioned at any location desired. The adaptor 1118 is configured to fluidly couple the gas supply tubing with an inner channel 1120 positioned within the first receptacle 1104. The inner channel 1120 may extend distally from a first end adjacent to the top portion 1114 of the container to a second end. The inner channel 1120 may be formed from a similar material as the container 1102 and may form a sub-chamber within the first receptacle 1104. In some embodiments, opposing sides of the container 1102 may be heat-sealed at the edges 1124 of the inner channel 1120 so that the inner channel 1120 is formed from the container 1102. The inner channel 1120 may further include a flow control mechanism 1126 disposed at a second end to control a flow of gas through the inner channel 1120 and prevent water 1134 from entering the inner channel 1120. Some illustrative flow control mechanisms 1126 may include, but are not limited to, duckbill valves, umbrella valves, hydrophobic membranes, etc. The flow control mechanism 1126 is configured to allow air/gas to pass from the inner channel 1120 to the first receptacle 1104 to pressurize the first receptacle 1104 while preventing water from flowing into the inner channel 1120 and/or the gas supply tube 240c.

FIG. 11B is an enlarged view of region B of FIG. 11A illustrating the inner channel 1120 formed with a heat-sealed edge 1124. In FIG. 11B, the bottom portion 1122 of the container 1102 is not illustrated to more particularly show how the opposing sides of the container 1102 are joined to form the inner channel 1120. As can be seen in FIG. 11B, opposing sides 1102a, 1102b of the container 1102 are brought together and heat-sealed to form the channel 1120. It is contemplated that other methods of securing the opposing sides 1102a, 1102b may be used, as desired. The flow control mechanism 1126 may be secured within the inner channel 1120 at a second end thereof.

Returning to FIG. 11A, the water supply tubing 245c extends from a second end external to the reservoir 1100 to a first end coupled to a second coupling mechanism or adaptor 1132. The second adaptor 1132 may be positioned adjacent to the bottom portion 1122 of the container 1102 so that fluid readily flows from the first receptacle to the water supply tubing 245c when the container 1102 is pressurized. A lumen extends through the water supply tubing 245c for receiving a flow of fluid therethrough. The lumen of the lens wash supply/irrigation supply tubing 245c is in selective operative fluid communication with the bottom portion of the container 1102. In the illustrated embodiment, the gas supply tubing 240c and the water supply tubing 245c may enter the container 1102 through separate adaptors 1118, 1132. However, in some embodiments, the water supply tubing 245c may enter through other parts of the container 1102 such as, but not limited to a top portion 1114 thereof. In such an instance, the water supply tubing 245c may include a dip tube which extends to the bottom portion 1122 of the container 1102.

A portion of the gas supply tubing 240c and a portion of the water supply tubing 245c may extend from the container 1102, and may be connected in fluid communication with the endoscope at gas/lens wash connection on the connector portion 265 of the umbilical. The portion of the gas supply tubing 240c is connected in fluid communication with a gas pump (not explicitly shown) and gas feed line (not explicitly shown), and the portion of the lens wash supply tubing 245c is connected in fluid communication with lens wash feed line (not explicitly shown), within connector portion 265. While not explicitly shown, irrigation supply tubing may be coupled to the container 1102 via a separate adaptor or port to supply irrigation fluid from the reservoir 1100.

It is contemplated that the reservoir 1100 may be filled and refilled as needed by removing the cap 1108 and pouring water into the first receptacle 1104. The refilling of the reservoir 1100 may be performed during a procedure or between procedures, as necessary. The water may be sterile or non-sterile, as desired. For example, sterile water may be used for therapeutic procedures while non-sterile water may be used for diagnostic procedures. It is contemplated that refilling the reservoir 1100 with sterile or non-sterile water may create more flexibility and reduce the need to have as much sterile water in storage. Further, refilling the reservoir 1100 via the port 1106 and removable cap 1108 may also remove the need to disconnect the reservoir 1100 from the tubing 240c, 245c throughout the day eliminating or greatly reducing the possibility of cross contamination by removing the need to replace the water container.

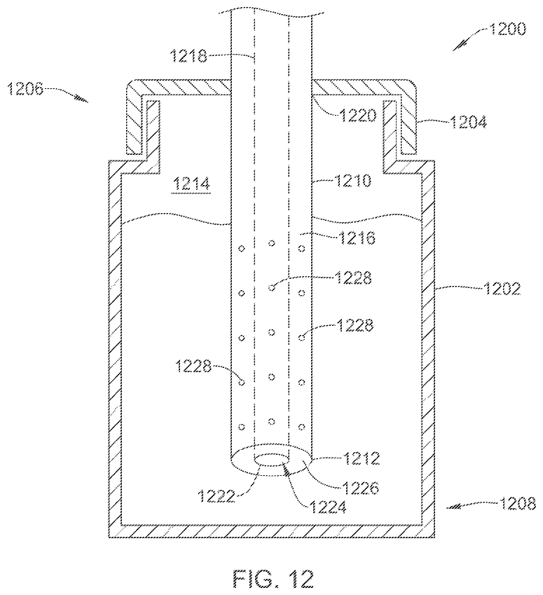

FIG. 12 depicts another illustrative reservoir 1200 for use with an endoscope system. The reservoir 1200 is arranged and configured for distributing fluid to an endoscope system. The system apart from the reservoir 1200 includes components similar to the endoscope and endoscope systems described with regard to FIGS. 1-4; however, not all features may be described or shown here if not pertinent to the fluid circuit of the system.

The fluid container 1202 is shown with reservoir top or cap 1204, which may be removably attachable to the top portion 1206 of the container 1202 (e.g., in a bottle and threaded cap arrangement). The cap 1204 may be removably attached in order to replenish fluid in the reservoir when it becomes depleted. Alternatively, the reservoir bottom and cap 1204 may be sealed to each other or they may be manufactured as a single, integral body (e.g., similar to a sealed monolithic rigid or semi-rigid bottle or softer IV bag or pouch). In such embodiments, a fill port may be included on another part of the reservoir for replenishing fluid.

A gas supply tubing 1210 extends from a second end external to the container 1202 to a first end 1212 adjacent to a bottom portion 1208 of the container 1202. The gas supply tubing 1210 may extend through an opening 1220 in the cap 1204 and into the container 1202 such that the gas supply tubing 1210 is in fluid communication with an interior 1214 container. A gasket or sealing member (not explicitly shown) may be positioned in the opening 1220 to provide a gas tight seal between the gas supply tubing 1210 and the cap 1204. A lumen 1216 extends through the gas supply tubing 1210 for receiving a flow of air and/or gas therethrough. The first end 1212 of the gas supply tubing 1210 may include a sealing member 1226. The sealing member 1226 may prevent gas from exiting the first end 1212 of the gas supply tubing 1210. Further, the sealing member 1226 may be configured to act as weight to maintain the first end 1212 of the gas supply tubing 1210 at or near the bottom portion 1208 of the container 1202.

The sidewall of the gas supply tubing 1210 may be configured to allow gas to pass from the lumen 1216 of the gas supply tubing 1210 into the container 1202 while water is inhibited from flowing from the container into the second lumen. For example, the gas supply tubing 1210 may include a plurality of apertures 1228 extending through a sidewall of the gas supply tubing 1210. The apertures 1228 may extend from an external surface to the internal surface of the gas supply tubing 1210 to fluidly couple the lumen 1216 with the interior 1214 of the container 1202. The plurality of apertures 1228 may be sized such that air may flow from the lumen 1216 of the gas supply tubing 1210 to the interior 1214 of the container 1202 but the surface tension of the water is sufficient to keep water from entering the lumen 1216. In some cases, the plurality of apertures 1228 may be considered to be pinholes. It is contemplated that the gas supply tubing 1210 may include any number of apertures 1228 desired. For example, the gas supply tubing 1210 may include one or more, five or more, ten or more, twenty or more, fifty or more apertures 1228. Further, the plurality of apertures 1228 may be uniformly or eccentrically distributed about a circumference and/or length of the gas supply tubing 1210. In some embodiments, the gas supply tubing 1210 may be formed from an elastomeric or deformable material which expands the size of the plurality of apertures 1228 as air pressure increases within the lumen 1216 and contracts the size of the plurality of apertures 1228 as air pressure decreases within the lumen 1216. Some illustrative materials for the gas supply tubing 1210 may include, but are not limited to, low density polyethylene (LDPE), high density polyethylene (HDPE), poly(vinyl alcohol) (PVA), silicone, polytetrafluoroethylene (PTFE), etc. In yet other embodiments, the plurality of apertures 1228 may be the gaps between filaments of a finely woven mesh.

A water supply tube 1218 may be co-axially disposed within the lumen 1216 of the gas supply tube 1210. The water supply tubing 1218 extends from a second end external to the container 1202 to a first end adjacent to a bottom portion 1208 of the container 1202. The first end 1222 of the water supply tubing 1218 and the first end 1212 of the gas supply tubing 1210 may be positioned at a similar location within the container 1202. The first end 1222 of the water supply tubing 1218 is in operative fluid communication with an interior 1214 of the container 1202. A lumen 1224 extends through the water supply tubing 1218 for receiving a flow of fluid therethrough. However, as the first end 1212 of the gas supply tubing 1210 is closed, water is precluded from entering the gas supply tubing 1210. The second ends of the gas supply tubing 1210 and the water supply tubing 1218 may be coupled to a manifold (if so provided) or a connector portion 265 of an endoscope system.

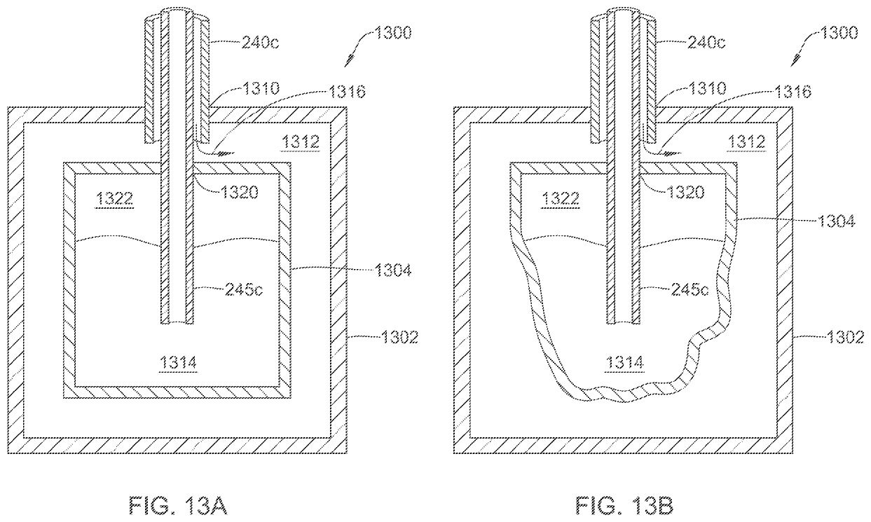

FIG. 13A depicts a cross-sectional side view of an illustrative fluid reservoir 1300 in a first configuration and FIG. 13B depicts a schematic side view of the illustrative reservoir 1300 of FIG. 13A in a second configuration. The reservoir 1300 may be configured to be used in an endoscopic system and includes components similar to the endoscope and endoscope systems described with regard to FIGS. 1-4; however, not all features may be described or shown here if not pertinent to the fluid circuit of the system. In the illustrated embodiment, a different means for performing insufflation may be required.

The reservoir 1300 may include an exterior container 1302 configured to hold a first fluid chamber 1304. A gas supply tubing 240c extends from a second end external to the reservoir 1300 to a first end adjacent an opening 1310 in the exterior container 1302 such that the gas supply tubing 240c is in fluid communication with an interior or a cavity 1312 of the exterior container 1302. A lumen extends through the gas supply tubing 240c for receiving a flow of air and/or gas therethrough. The exterior container 1302 fluidly isolates the air/gas received from the gas supply tubing 240c from the water 1314 in the first chamber 1304. The exterior container 1302 may be rigid so that the exterior container 1302 resists expansion and increases the pressures of the cavity 1312 as air/gas flows into the cavity 1312, along flow path 1316. In the absence of a positive air flow, the pressure within the cavity 1312 may dissipate or the pressure may be maintained. It is contemplated that the exterior container 1302 may include a one-way valve disposed at or adjacent an inlet of the cavity 1312. Examples of one-way valves include various check valves described above. The one-way valve may prevent air from exiting the exterior container 1302 even in the absence of a positive air flow.

A lens wash supply tube or shared water supply tube (e.g. supplies water for both lens wash and irrigation) 245c extends from a second end external to the reservoir 1300 to a first end adjacent an opening 1320 in the first chamber 1304 such that the water supply tubing 245c is in operative fluid communication with an interior or a cavity 1322 of the first chamber 1304. The water supply tube 245c may extend through the gas supply tube 240c such that the longitudinal axis of the water supply tube 245c is co-axial with the longitudinal axis of the gas supply tube 240c. In other examples, the water supply tube 245c and the gas supply tube 240c may extend such that the longitudinal axes thereof are laterally offset. A lumen extends through the water supply tubing 245c for receiving a flow of fluid therethrough. As air enters the exterior container 1302, the pressure within the cavity 1312 of the exterior container 1302 increases and applies a pressure to the first chamber 1304, as shown in FIG. 13B. As the pressure of the exterior container 1302 increases, the first chamber 1304 may be compressed causing water 1314 within the first chamber 1304 to be expelled up the water supply tube 245c and to the endoscope for lens wash and/or irrigation.

The first chamber 1304 may be formed from a lightweight, flexible material that does not necessarily stretch, such as, but not limited to low density polyethylene (LDPE), thermoplastic polyurethane (TPU), silicone, polyethylene terephthalate (PET), aluminum, nylon, polyethylene (PE), or combinations thereof, etc.

A portion of a gas supply tubing 240c and a portion of water supply tubing 245c may be connected in fluid communication with the endoscope at gas/lens wash connection on the connector portion 265 of the umbilical. The gas supply tubing 240c is connected in fluid communication with a gas pump (not explicitly shown) and gas feed line (not explicitly shown), and the water supply tubing 245c is connected in fluid communication with lens wash feed line (not explicitly shown), within connector portion 265. In some examples, the gas supply tubing 240c may include a manifold to fluidly couple portions of the gas supply tubing 240c. Similarly, the lens wash supply tubing 245c may include a manifold to fluidly couple portions of the lens wash supply tubing with the shared lens wash/irrigation (or water) supply tubing 245c. While not explicitly shown, irrigation supply tubing may be coupled to the manifold, if so provided, to supply irrigation fluid from the reservoir 1300. In other cases, a separate irrigation supply tube may be provided.

As will be appreciated, the lengths of irrigation, lens wash, gas supply, alternate gas supply tubing may have any suitable size (e.g., diameter). In addition, the sizing (e.g., diameters) of the tubing may vary depending on the application. In one non-limiting embodiment, the irrigation supply tubing may have an inner diameter of approximately 6.5 mm and an outer diameter of 9.7 mm. The lens wash supply tubing may have an inner diameter of approximately 5 mm and an outer diameter of 8 mm. The gas supply tubing may have an inner diameter of approximately 2 mm and an outer diameter of 3.5 mm. The alternative gas supply tubing may have an inner diameter of approximately 5 mm and an outer diameter of 8 mm.

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

All apparatuses and methods discussed herein are examples of apparatuses and/or methods implemented in accordance with one or more principles of this disclosure. These examples are not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure.

In the foregoing description and the following claims, the following will be appreciated. The phrases “at least one”, “one or more”, and “and/or”, as used herein, are open-ended expressions that are both conjunctive and disjunctive in operation. The term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The foregoing discussion has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. One skilled in the art will appreciate that the disclosure may be used with many modifications of structure, arrangement, proportions, materials, components, and otherwise, used in the practice of the disclosure, which are particularly adapted to specific environments and operative requirements without departing from the principles of the present disclosure. For example, elements shown as integrally formed may be constructed of multiple parts or elements shown as multiple parts may be integrally formed, the operation of elements may be reversed or otherwise varied, the size or dimensions of the elements may be varied, and features and components of various embodiments may be selectively combined. The presently disclosed embodiments are therefore to be considered in all respects as illustrative and not restrictive, the scope of the claimed invention being indicated by the appended claims, and not limited to the foregoing description.

The following claims are hereby incorporated into this Detailed Description by this reference, with each claim standing on its own as a separate embodiment of the present disclosure. In the claims, the term “comprises/comprising” does not exclude the presence of other elements or steps. Furthermore, although individually listed, a plurality of means, elements or method steps may be implemented by, e.g., a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second”, etc., do not preclude a plurality. Reference signs in the claims are provided merely as a clarifying example and shall not be construed as limiting the scope of the claims in any way.

1. A container and tube set arranged and configured to couple to an endoscope for use in an endoscopic procedure, the container and tube set comprising:
a container configured to contain a fluid, the container having a bottom portion and a top portion;
a water supply tube including a first end, a second end, and a first lumen extending therethrough, wherein the first lumen is in selective fluid communication with the bottom portion of the container and the second end of the water supply tube is positioned external to the container;
a gas supply tube including a first end, a second end, and a second lumen extending therethrough, wherein the second lumen is in operative fluid communication with the container and the second end of the gas supply tube is positioned external to the container; and
a weight coupled to the first end of the water supply tube and a first end of the gas supply tube, the weight comprising a housing having a housing lumen extending from a first end of the housing to a second end of the housing.

2. The container and tube set of claim 1, further comprising one or more apertures extending through a side wall of the housing, the one or more apertures positioned between the first and second ends of the housing.

3. The container and tube set of claim 1, wherein the housing lumen has a cross-sectional dimension that incrementally decreases from the first end to the second end.

4. The container and tube set of claim 3, wherein a first transition in the cross-sectional dimension of the housing lumen defines a first ledge and the first end of the gas supply tube is configured to abut the first ledge.

5. The container and tube set of claim 4, wherein the one or more apertures are positioned between the first ledge and the second end of the housing.

6. The container and tube set of claim 4, wherein a second transition in the cross-sectional dimension of the housing lumen defines a second ledge and the first end of the water supply tube is configured to abut the second ledge.

7. The container and tube set of claim 2, wherein a flow of gas through the second lumen is configured to exit the one or more apertures.

8. The container and tube set of claim 1, wherein a flow of water is configured to enter the first lumen through the second end of the housing upon pressurization of the container.

9. The container and tube set of claim 2, further comprising a one-way valve coupled to the one or more apertures.

10. A container and tube set arranged and configured to couple to an endoscope for use in an endoscopic procedure, the container and tube set comprising:
a container configured to contain a fluid, the container having a bottom portion and a top portion;
a water supply tube including a first end, a second end, and a first lumen extending therethrough, wherein the first lumen is in selective fluid communication with the bottom portion of the container and the second end of the water supply tube is positioned external to the container;
a gas supply tube including a first end, a second end, and a second lumen extending therethrough, wherein the second lumen is in operative fluid communication with the container and the second end of the gas supply tube is positioned external to the container; and
a weight coupled to the first end of the water supply tube and a first end of the gas supply tube, the weight comprises a housing having a first housing lumen extending from a first end of the housing to a second end of the housing and a second housing lumen extending at a non-orthogonal angle relative to the first housing lumen and through a side wall of the housing.

11. The container and tube set of claim 10, further comprising one or more apertures formed in the second end of the housing.

12. The container and tube set of claim 10, wherein the first housing lumen has a cross-sectional dimension that incrementally decreases from the first end to the second end.

13. The container and tube set of claim 12, wherein a first transition in the cross-sectional dimension of the housing lumen defines a first ledge and the first end of the gas supply tube is configured to abut the first ledge.

14. The container and tube set of claim 13, wherein a first opening of the second housing lumen is positioned between the first ledge and the second end of the housing and a second opening of the second housing lumen is positioned adjacent to the second end of the housing.

15. The container and tube set of claim 11, wherein the first lumen of the water supply tube is in fluid communication with the second housing lumen and the second lumen of the gas supply tube is in fluid communication with the one or more apertures.

16. The container and tube set of claim 11, further comprising a one-way valve coupled to the one or more apertures.

17. A container arranged and configured to couple to an endoscope for use in an endoscopic procedure, the container comprising:
a flexible container configured to contain a fluid within a first receptacle thereof, the container having a bottom portion and a top portion;
a water outlet positioned adjacent to the bottom portion of the container; and
a gas inlet, the gas inlet in fluid communication with a second receptacle of the container;
wherein the second receptacle comprises a hydrophobic membrane.

18. The container of claim 17 wherein the hydrophobic membrane is configured to allow gas to pass from the second receptacle to the first receptacle and to preclude a passage of water from the first receptacle to the second receptacle.

19. The container of claim 17, further comprising:
a water supply tube including a first end, a second end, and a first lumen extending therethrough, wherein the first lumen is in fluid communication with the first receptacle in the bottom portion of the container and the second end of the water supply tube is positioned external to the container; and
a gas supply tube including a first end, a second end, and a second lumen extending therethrough, wherein the second lumen is in operative fluid communication with the first receptacle and the second end of the gas supply tube is positioned external to the container.

20. The container of claim 17, further comprising a port positioned adjacent to the top portion of the container, wherein the port is configured to selectively fluidly couple the first receptacle of the container with an external water source; and
a removable cap selectively coupled to the port.
